(19) United States
(12) Patent Application Publication
(10) Pub. No.: US 2014/0050668 A1
(43) Pub. Date: Feb. 20, 2014

(54) VISUALIZATION OF LIPID METABOLISM

(71) Applicants: Oliver Bruns, Boston, MA (US); Heinrich Hohenberg, Hamburg (DE); Rudolph Reimer, Hamburg (DE); Ulrich Tromsdorf, Plankstadt (DE); Horst Weller, Hamburg (DE); Gerhard Adam, Hamburg (DE); Harald Ittrich, Hamburg (DE); Michael Kaul, Hamburg (DE); Peter Nielsen, Besitz (DE); Barbara Freund, Koln (DE); Alexander Bartelt, Hamburg (DE); Jörg Heeren, Hamburg (DE)

(72) Inventors: Oliver Bruns, Boston, MA (US); Heinrich Hohenberg, Hamburg (DE); Rudolph Reimer, Hamburg (DE); Ulrich Tromsdorf, Plankstadt (DE); Horst Weller, Hamburg (DE); Gerhard Adam, Hamburg (DE); Harald Ittrich, Hamburg (DE); Michael Kaul, Hamburg (DE); Peter Nielsen, Besitz (DE); Barbara Freund, Koln (DE); Alexander Bartelt, Hamburg (DE); Jörg Heeren, Hamburg (DE)

(73) Assignees: UNIVERSITAETSKLINIKUM HAMBURG-EPPENDORF, Hamburg (DE); HEINRICH-PETTE-INSTITUT LEIBNIZ-INSTITUT FUER EXPERIMENTELLE VIROLOGIE STIFTUNG BUERGERLICHEN RE, Hamburg (DE); CENTRUM FUR ANGEWANDTE NANOTECHNOLOGIE (CAN) GMBH, Hamburg (DE)

(21) Appl. No.: 13/980,751

(22) PCT Filed: Jan. 20, 2012
(86) PCT No.: PCT/EP12/50863

§ 371 Date: Nov. 5, 2013

Related U.S. Application Data
(60) Provisional application No. 61/435,110, filed on Jan. 21, 2011.

(51) Int. Cl. A61K 49/14

(52) U.S. Cl. CPC A61K 49/14
USPC 424/9.32

(57) ABSTRACT

The present invention relates to the field of in vivo determination of enzyme activity. It also allows visualization of organisms, organs, tissues and cells. In particular, the present invention provides a method of in vivo visualization and a composition suitable for in vivo determination and/or visualization of enzyme activity by methods such as Magnetic Resonance Imaging, also called Magnetic Resonance Tomography (MRI or MRT), or Magnetic Particle Imaging (MPI). In particular, the activity of the enzyme lipoprotein lipase affects the signals received and allows conclusions on the lipid metabolism of an organism, an organ system, an organ, a tissue and a cell of interest, This method can be employed, e.g., for diagnosis of cardiac disorders, of tumor prognosis and of disorders of the lipid metabolism. The composition used comprises superparamagnetic iron oxide nanocrystals (SPIO) incorporated in the core of lipid micelles designated nanosomes.

The present invention relates to the field of in vivo determination of enzyme activity. It allows visualization of organisms, organs, tissues and cells. In particular, the present invention provides a method of in vivo visualization and a composition suitable for in vivo determination and/or visualization of enzyme activity by methods such as Magnetic Resonance Imaging, also called Magnetic Resonance Tomography (MRI or MRT), or Magnetic Particle Imaging (MPI). In particular, the activity of the enzyme lipoprotein lipase affects the signals received and allows conclusions on the lipid metabolism of an organism, an organ system, an organ, a tissue and a cell of interest. This method can be employed, e.g., for diagnosis of cardiac disorders, of tumor prognosis and of disorders of the lipid metabolism. The composition used comprises superparamagnetic iron oxide nanocrystals (SPIO) incorporated in the core of lipid micelles designated nanosomes.

In contrast to glucose, lipids such as triacylglycerol (TAG) or cholesterolesters (CE) are not soluble in the blood and are transported in the form of triglyceride-rich lipoproteins (TRL). These micelles comprise an amphiphillic monolayer of phospholipids and free cholesterol in which apolipoproteins are embedded, e.g., apoE. In the hydrophobic core, TAG and CB are found. In the intestine, lipids are packaged into lipoproteins as chylomicrons, and are transported to peripheral tissues such as adipose tissue, heart and muscle. In the bloodstream, lipoprotein lipase (LPL) mediates the release of fatty acids from TAG. While the fatty acids are taken up by underlying tissues, the remaining rather cholesterol-rich chylomicron remnant particles are cleared by the liver (Williams 2008). The liver can generate endogenous lipoproteins, Very-Low-Density lipoproteins, VLDL, when uptake of lipids from food is low. These can be taken up analogously to the chylomicrons by the lipid-consuming tissues. Further lipoprotein fractions, high-density lipoproteins (HDL) and low-density lipoproteins (LDL) also carry a small fraction of TAG.

The enzyme lipoprotein lipase, LPL, is localized at the endothelium of cells taking up lipids, in particular, heart and skeletal muscle as well as white and brown adipose tissue. LPL catalyses the reaction of triacylglycerol+H2O<=>diacylglycerol+a carboxylate. Its main role lies in hydrolys of triacylglycerols in chylomicrons and very low-density lipoproteins (VLDL). It also hydrolyzes diacylglycerol. LPL is the gatekeeper enzyme in lipid metabolism, as it catalyses the most time critical step of lipolysis, and an inhibition of LPL is thus sufficient for blocking lipid uptake in tissue. LPL is the central enzyme in vascular TAG and fatty acid metabolism (Merkel 2002; Olivacrona 2010). A defect in its gene leads to a severe hypertriglyceridemia with pancreatitis as clinical consequence in humans. LPL activity is crucial for heart function as most of energy consumed by the heart is produced by oxidation of lipoprotein-derived fatty acids.

Mice which are deficient for LPL in the heart develop cardiac dysfunction despite an increased glucose oxidation (Augustus 2006; Yamashita 2008). Therefore, changes in LPL expression in type 2 diabetics might be one reason for the heart failure which frequently occurs in these patients (Park 2007). Overexpression of the enzyme also leads to cardiomyopathy. Thus, controlled activity of LPL is essential for physiological heart function.

Growing tumors secrete pro-inflammatory cytokines like IL-6 and TNFalpha. These cytokines down-regulate the expression and activity of LPL in peripheral tissues. As LPL is crucial for lipid uptake, decreasing its activity results in a marked caloric deficit in adipose tissue, muscle and heart. The consequence is a massive loss of muscle and fat mass which ultimately leads to cachexia. On the other hand, reports found a link between high expression of LPL by certain cancer tumor cells such as non-small cell lung and a shorter patient survival (Trost 2009). The same correlation of high LPL expression and poor clinical outcome was found in chronic lymphocytic leukemia (Heintel 2005; Oppezzo 2005). Taken together, these studies suggest an important role for LPL activity in tumor development and associated cachexia, as it delivers energy for tumor growth while it steals energy from peripheral tissues.

Magnetic resonance imaging (MRI), nuclear magnetic resonance imaging (NM), or magnetic resonance tomography (MRT) is a medical imaging technique used to visualize detailed internal structures in vivo. The terms are used interchangeably in the context of this application. The good contrast MRI provides between the different soft tissues of the body makes it especially useful in brain, muscles, heart, and cancer.

MRI has a growing importance in diagnosing heart function. It can be used to visualize cardiac anatomy with high resolution. So called Cine sequences, which visualize the heart cycle, allow determination of the ejection volume and movement of the heart muscle. Use of contrast agent also allows visualization of perfusion of the heart. A so-called “Late-enhancement” in MRI imaging with contrast agent can show scars e.g. caused by infarction.

MRI uses a magnetic field to align the magnetization of some atoms in the body, then uses radio frequency fields to systematically alter the alignment of this magnetization. This causes the nuclei to produce a rotating magnetic field detectable by the scanner, which is recorded to visualize the scanned area of the body.

MRI contrast agents may be injected intravenously to enhance the visibility of internal body structures, e.g., of blood vessels or tumors. MRI contrast agents alter the relaxation times of tissues and body cavities where they are present. Depending on the image weighting, this can give a higher or lower signal.

The most commonly used compounds for contrast enhancement are gadolinium-based. LDL- and HDL-like micelles enriched with hydrophobic gadolinium chelates have been used as contrast agent for the detection of tumors and atherosclerotic plaques (Frias et al., 2004; Corbin et al., 2006; Glickson et al., 2008). However, these agents have some disadvantages.

Patients with renal disorders or insufficiency have shown serious side effects upon use of gadolinium-based contrast agents, some fatal. Due to these problems, these contrast agents should not any more be used in such patients. Renal disorders are most prevalent either in older people or in patients suffering from diabetes and high blood pressure. As this is exactly the group of patients for which MRT visualization of, e.g., the heart is of the highest interest, alternative MRT contrast agents are needed.

An alternative, which does not show serious side effects and can be used in all patients, is provided by iron oxide based MRI contrast agents. Two types iron oxide contrast agents are well known in the state of the art: Superparamagnetic Iron Oxide (SPIO) and Ultrasmall Superparamagnetic Iron Oxide (USPIO). These contrast agents typically are suspended colloids of iron oxide nanoparticles. When injected during imaging, they reduce the T2 signals of absorbing tissues. SPIO and USPIO contrast agents have been used successfully, e.g., for liver tumor enhancement.

Due to their excellent suitability for electron microscopy as well as fluorescence and magnetic-resonance-imaging, quantum dots, gold and superparamagnetic iron oxide (SPIOs) nanocrystals have been extensively applied as labels for biomedical imaging (Michalet 2005; Alivisatos 2004). Furthermore, several sensors based on nanocrystals for different applications have been developed over the last years (Perez 2002; Medintz 2005; Koh 2008; Lee 2008; Snee 2008; McLaurin 2009). But none of these sensors has been applied in vivo so far.

MRI or the related method of Magnetic Particle Imaging (MPI) can be used for visualizing physical function, e.g., of the heart as well as perfusion. However, biochemical parameters such as the energy metabolism of specific areas cannot yet be visualized in vivo with acceptable resolution. This could be of particular benefit, e.g., because it would allow for diagnosis of a future potential loss of function of tissue, e.g., by showing change from lipid to glucose metabolism for energy generation or excessive use of lipids. This would allow for therapeutic intervention and could prevent chronic loss of function.

Previously, the inventors (Bruns et al., Nature Nanotechnology, 2009, which is incorporated herein by reference) disclosed a new method to visualize lipoproteins, using superparamagnetic iron oxide nanocrystals embedded into the core of lipoproteins. They showed that it is possible to image and quantify the kinetics of lipoprotein metabolism in vivo using dynamic MRI. The lipoproteins were taken up by liver cells in wild-type mice, and displayed defective clearance in knock-out mice lacking a lipoprotein receptor or its ligand, indicating that the nanocrystals did not influence the specificity of the metabolic process. Using this strategy, it is possible to study the clearance of lipoproteins in metabolic disorders and to improve the contrast in clinical imaging. However, no dependence of enzymatic activity was observed in the published experiments.

In light of this, the inventors now solved the problem of providing an in vivo method of determining and visualizing a compartment in a subject dependent on enzymatic activity in said compartment, in particular, lipoprotein lipase (LPL) activity. Thus, LPL activity can be determined and visualized in vivo. The invention is further described below and in the appended claims.

The invention provides a method of in vivo visualization, comprising
a) administering a composition comprising superparamagnetic iron oxide nanocrystals (SPIO) incorporated in the core of nanosomes to a subject, and
b) determining and visualizing lipoprotein lipase activity in a compartment of the subject.

The invention also provides a method of in vivo determination and/or visualization of lipoprotein lipase (LPL) activity, comprising
a) administering a composition comprising superparamagnetic iron oxide nanocrystals (SPIO) incorporated in the core of nanosomes to a subject, and
b) determining and visualizing presence of SPIO in a compartment of the subject, wherein the presence of SPIO indicates lipoprotein lipase (LPL) activity in the compartment or associated with the compartment.

The invention also provides a composition comprising superparamagnetic iron oxide nanocrystals (SPIO) incorporated in the core of nanosomes for use in in vivo visualizing lipoprotein lipase (LPL) activity, comprising
a) administering the composition to a subject, and
b) determining and visualizing presence of SPIO in a compartment of the subject, wherein the presence of SPIO indicates lipoprotein lipase (LPL) activity in the compartment.

A composition comprising superparamagnetic iron oxide nanocrystals (SPIO) incorporated in the core of nanosomes (SPIO nanosomes) can be prepared, e.g., as disclosed below or according to methods disclosed by Bruns et al., Nature Nanotechnology 2009, or according to Tromsdorf et al, Nano Letters 2007.

The nanosomes may be prepared from biological samples, e.g., from the subject which is to be examined. For example, lipids used for the preparation of the nanosomes can be extracted from lipoproteins isolated by standard centrifugation protocols from plasma.

The nanosomes may also be artificially prepared, e.g., assembled from the components according to methods known in the state of the art.

In one embodiment, the nanosomes are based on TEL such as chylomicrons or VLDL. It is also possible that the nanosomes are based on the composition of, e.g. LDL or HDL.

Chylomicrons are synthesized in the postprandial phase by enterocytes within the intestine and have a diameter between 75-1200 nm depending on the composition of the meal. The size of SPIO-nanosomes is dependent on the lipid mixture used, e.g., for lipids extracted from human plasma TEL, it is approximately 250 nm and therefore is within the size of physiological postprandial lipoproteins.

After assembling within intestinal cells, chylomicrons enter the blood stream via the thoracic duct, which is the largest lymphatic vessel in the body draining into the systemic circulation via the left subclavian vein into the heart. Similar to chylomicrons, intravenously injected nanosomes reach the systemic circulation via the heart. In addition, it is important to note that nascent chylomicrons do not contain any apolipoprotein E (apoE) or lipoprotein lipase (LPL). Consequently, nanosomes do not need to contain exogenously added apoE and LPL when serving as a model particle for chylomicrons.

Therefore, SPIO-Nanosomes can be prepared with or without the addition of apolipoproteins. If the apolipoproteins are not added to nanosomes before administration to the subject, e.g., i.v., the nanosomes will acquire apolipoproteins after the Injection into the circulation.

In one embodiment, the nanosomes are prepared comprising apolipoproteins such as ApoB. Apolipoproteins may be of human or other origin, e.g., mouse, rat, ape or swine. Preferably, they are of the same species origin as the subject. The subject may, e.g., be human, mouse, rat, ape or swine. The composition of the nanosomes may vary, depending on the intended organ or tissue that is to be analysed. In particular, apolipoproteins or a particular lipid composition may be chosen to target the nanosomes to specific organs/tissues.

Nanosomes are micelles made of, e.g., lipids extracted from lipoproteins, and lipophilic nanocrystals may be embedded in the core.

In one embodiment, SPIO nanosomes may comprise at least about 40% triglycerides, They may comprise about 0.25% to about 20% phospholipids, about 0% - about 20% cholesterin and/or cholesterin ester, about 0% - about 10% cholate and 0% - about 50% dryweight of nanocrystals, preferably, 3% to about 30% dryweight of nanocrystals. The nanosomes preferably have a size between 30 nm-2 μm. % in the context of the application relates to weight/weight, if not explicitely mentioned otherwise.

The composition comprising nanosomes preferably comprises about 0.1% - about 30% lipids in total in an aqueous buffer. Preferably, the composition is non-toxic and suitable for administration to a human, e.g., for i.v. injection.

Preferably, nanosomes which may be used in the context of the invention carry lipophilic nanocrystals (also designated nanoparticles) which may cause a detectable signal in imaging modality used for humans, e.g. SPIO nanocrystals of about 2-30 nm size, specifically, about 6 nm or about 10 nm for MRI, and they are a substrate for LPL.

The SPIO preferably comprise nanocrystals having a size of about 2 to about 30 nm, or about 5 to about 20 nm, e.g., about 6 to about 10 nm. The SPIO preferably comprise Fe₃O₄ and/or Fe₂O₃ nanocrystals (e.g, having a size of about 6-10 nm), but they may also or additionally comprise MnFe₂O₄ nanocrystals. Alternatively or additionally, other kinds of superparamagnetic nanoparticles or superparamagnetic materials with a size less than 50 nm can be used in the nanosomes of the invention. These materials could include superparamagnetic iron nanoparticles with a gold shell (iron-gold core-shell nanoparticles), superparamagnetic iron nanoparticles with an iron oxide shell, superparamagnetic iron platin nanoparticles, superparamagnetic iron oxide nanocrystals with another composition than Fe₃O₄ or Fe₂O₃. Any material which can be used to cause a contrast in MRI pictures may be used.

All kinds of lipophilic nanocrystals, like quantum dots, SPIO or gold nanoparticles, preferably with particle sizes between 2 and 30 nm, can be embedded into the nanosomes used in the invention. The nanosomes comprising the nanocrystals allow multimodal visualization as well as quantification of lipoprotein metabolism, in particular, LPL activity, in real-time by non-invasive imaging in vivo. The MRI contrast agent based on nanocrystals used in the present invention may consist of nanosomes with multiple SPIOs inside, These SPIO form an ensemble in which their magnetic moments interacts with each other, This interaction leads to a maximized and constant r2* relaxivity which can be described by the static dephasing regime (SDR). It allows quantifying lipoprotein metabolism by real-time MR imaging (Bruns 2009).

In the method of the invention, visualization of the SPIO nanosomes takes place in vivo, i.e. in a compartment of an organism (a subject). The compartment may be an organ, a tissue or a cell or an area thereof. The LPL enzyme activity may be in a compartment or associated with the compartment. For example, the LPL enzyme activity is usually associated with the endothelium of a tissue/organ. This is enzyme activity is not considered to be associated with the circulation, but with the tissue/organ bordering the circulation, i.e., the compartment into which the SPIO are taken up.

For the purposes of this application, the circulation is thus not considered a compartment or organ. LPL activity in the tissues/compartments leads to diminished presence of SPIO nanosomes in the circulation, as these can be taken up from the circulation into organs/tissues dependent on LPL activity.

In general, in the context of the invention, “a” or “the” does not only designate “one”, but also includes a plurality. For example “a compartment” may also be more than one compartment. The method of the invention allows a high spatial resolution of the presence of the SPIO.

In the context of the invention, a tissue may be selected from the group comprising tumor, atherosclerotic plaque, sites of inflammation and adipose tissue. Adipose tissue may be white adipose tissue or brown adipose tissue. The organ or tissue analyzed by the method of the invention may express lipoprotein lipase and exhibit lipoprotein lipase activity under physiological or non-physiological conditions.

The organ is preferably selected from the group comprising heart, skeletal muscle, brain and tumors as well as sites of inflammation Liver and spleen are under physiological conditions not among these organs, as uptake into these organs is not LPL dependent. In conditions of tumors in the liver or spleen with an increased abnormal expression of LPL, the invention might be applied to measure LPL activity.

Administration of the SPIO nanosomes may be by oral or Intravenous administration, in particular, intravenous administration. Oral administration of nanosomes comprising contrast agents such as SPIO may be used to analyse, determine and/or localize sites of lipase activity in the gastrointestinal tract, in particular, pancreatic lipase activity.

In the context of the invention, the composition may be administered in an effective amount, i.e., an amount that allows visualization. This can be determined, e.g., by the clinician. In one embodiment about 0.05 mg iron within nanosomes/kg body weight-2 mg/kg body weight are to be administered intravenously. It may be favourable to use 0.1 mg iron within nanosomes/kg body weight-1 mg/kg body weight to be administered intravenously. Preferentially, 0.3 mg iron within nanosomes/kg body weight-0.5 mg/kg body weight are to be administered intravenously. The amount can be adapted depending on, e.g., age, sex, the aim of the analysis and/or the condition of the subject.

The determination and/or visualization may be performed by MRI. Dynamic MRI, may also be employed. Suitable protocols are described herein or in Bruns et al., Nature Nanotechnology 2009. One significant advantage of the method of the invention is that it may be performed without invasive methods. Another advantage is that no administration or radioactive compounds to the subject are required.

In addition, the invention may be used to determine LPL activity with other non-invasive imaging techniques in which lipophilic nanoparticies or nanocrystals can be used as a contrast agent. In particular, SPIO-nanosomes may be applied to contrast compartments and/or visualize and/or measure LPL activity by magnetic particle imaging (MN). For example, the heart might be contrasted, and/or LPL activity may be visualized in the heart by magnetic particle imaging (MPI),

The method of the invention may also be employed for visualization of the organ, tissue or cell. The SPIO nanosomes may be used similarly to a conventional contrasting agent. For example, as the agent is mostly taken up in perfused areas of a tissue or an organ, perfusion can be visualized.

In one embodiment of the method of the invention, the tissue that is visualized Is tumor tissue, and the LPL activity is predictive of progression of the tumor. In particular, a high LPL activity is predictive of fast progression of the tumor.

In one embodiment, the invention provides a method of diagnosing a tumor. The method may provide a prognosis of clinical outcome. In this context, a high lipase activity has been shown to correlate with a bad prognosis, i.e. fast progression of the tumor, and a low lipase activity correlates with slow progression of the tumor and/or regression. A higher lipase activity thus correlates with a worse prognosis.

In one embodiment, the invention provides a method of diagnosing a disorder of the lipid metabolism, comprising performing the method of the invention. Disorders of the lipid metabolism may include type II diabetes and cachexia, Also, genetic deficiency for LPL, Apolipoprotein CII, Apolipoprotein AV and/or glycosylphosphatidylinositol-anchored high-density lipoprotein-binding protein 1 may be diagnosed by performing the method of the invention,

The invention also provides a method of diagnosing a cardiac disorder, comprising performing the method of the invention. The cardiac disorder may be selected from the group comprising coronary heart disease, coronary artery disease, cardiomyopathy, alcoholic cardiomyopathy, congenital heart disease, ischemic cardiomyopathy, hypertensive cardiomyopathy, nutritional diseases affecting the heart, valvular cardiomyopathy, inflammatory cardiomyopathy, cardiomyopathy secondary to a systemic metabolic disease or myocardiodystrophy. The following cardiac disorders may also be diagnosed: dilated cardiomyopathy, hypertrophic cardiomyopathy, arrhythmogenic right ventricular cardiomyopathy, restrictive cardiomyopathy or noncompaction cardiomyopathy. The method of the invention is especially advantageous for localizing disorders in specific areas of the heart and/or quantifying the area of an organ such as the heart which is afflicted by the disorder.

In one embodiment of the method of the invention, the organ that is visualized is heart. For example, viability and/or perfusion of an organ such as heart may be detected, e.g., scarring due to infarction. The method of the invention may also be used for diagnosing disorders of the lipid metabolism in the heart. For example, the organ that is visualized may be heart, and disorders of the lipid metabolism in the heart may be detected and/or localized to specific areas of the heart. Exemplary disorders are mentioned above.

In a method of diagnosis of the invention, one step may be comparing the results of the analysis of the subject with a reference, such as results from a healthy subject and/or a from a group of healthy subjects (i.e., average results from such a group) and/or from a subject or subjects previously diagnosed with the disorder or tumor in question. E.g., a result significantly varying from a healthy reference may indicate that the subject has a disorder.

The invention also relates to a diagnostic and/or prognostic composition comprising superparamagnetic iron oxide nanocrystals (RIO) incorporated in the core of nanosomes for use in in vivo determining and/or visualizing lipoprotein lipase (LPL) activity. The composition is also for use in diagnosing disorders of the lipid metabolism, for use in diagnosing cardiac disorders or for use in prognosis of a tumor, as described in detail above.

The inventors have demonstrated that it is possible to visualize enzyme activity in the heart and other tissues with the method of the invention. They provide a method for imaging of the metabolism with a high resolution, e.g., in the heart or in tumors, The method's results are comparable with PET-CT (Positron emission tomography combined with computer tomography) analysis with radioactive 18-Fluor-desoxyglucose (FDG), however, there is no requirement for use of radioactive isotopes (as in PET and SPECT) or ionising radiation (as in CT and X-rays). Furthermore, the metabolism is directly visualized with high-resolution MRI significantly improving spatial resolution compared to approaches based on PET or SPECT. In comparison to new development of systems like PET-MRT, the method of the invention still has the advantage of higher resolution combined with the option of not using radioactive tracers, Furthermore, apparatus requirements are lower (MRT only instead of PET-CT or PET-MRT).

Visualization, in the context of the invention, is meant to comprise steps of determining a result, e.g., a measure of LPL activity, and of generating an Image based on this result, wherein the activity is linked to an area in which it has been detected, and graphically represented. MRI or MPI are typical methods comprising visualization of a result in the context of the invention.

It is noted that the combined time and spatial resolution of the method of the invention are of excellent quality and could not be achieved with any other technique, neither by non-invasive nor invasive approaches. MRI, used with the method of the invention, can provide a spatial resolution of below 1 mm, preferably, of below 0.1 mm. Temporal resolution preferably is below 0.1 sec.

In the examples below, it is shown in a mouse model that in vivo imaging of uptake of SPIO nanosomes is feasible, and that Increased LPL activity can be detected via an increased uptake of SPIO nanosomes. Surprisingly, the uptake was directly dependent on LPL activity, as inhibition of LPL via THL, Tetrahydrolipstatin, specifically inhibited uptake. Quantitative analysis of the organ distribution with radioactive labelling of the SPIO in the nanosomes confirmed these result. The use of radioactive SPIO is however not required in the method of the invention.

The examples shown below are meant to illustrate, but not to limit the invention. Other embodiments can be envisaged by the skilled person taking the description of the invention into account.

### FIGURE LEGENDS

FIG. 1. Cold exposure modulates fasting and postprandial triglyceride-rich lipoprotein levels
(a) Triglyceride and (b) cholesterol FPLC profiles of pooled plasma from fasted FVB mice after 4 h and 24 h cold exposure at 4° C. (e) Plasma triglycerides during an oral fat tolerance test in control and cold-exposed FVB mice. (d) FPLC lipoprotein profiling in control and cold-exposed FVB mice two hours after an oral fat load. (e) Organ distribution of triolein-derived ³h-radioactivity in control and cold-exposed FVB mice two hours after gavage. Mean values ±s.e.m. with n=12 in (c) and n=4 in (e).*P<0.05; ^{&}P<0.01; ^{$}P<0.001.

FIG. 2. Activated BAT is a central target organ for TRL uptake
(a) Plasma clearance of ⁵⁹Fe-SPIO and (b)³H-triolein-labeled TRL in control and cold-exposed C57BL/6 mice. (c) Organ distribution of ⁵⁹Fe-SPIO and (d) triolein-derived ³H-radioactivity 15 min after intravenous injection. Mean values ±s.e.m. with n=5. (e) Representative transversal magnetic resonance (MR) images of a control and cold-exposed wild-type FVB mouse before and approximately 10 min after injection of SP10-labeled TRL. Arrows in upper panel point to BAT whereas arrows in lower panel indicate the liver. Bar: 1 cm. (f) Coronary MR images of a representative cold mouse before, 10 min and (g) 1 week after injection of SPIO-TRL with identical MR settings. (h) Representative intravital confocal microscopy images of dissected BAT in a live cold-exposed FVB mouse 2 min (left) and 30 min (right) after QD-TRL (green) injection (arrows indicate QD-TRL). FITC-dextran (red) to stain blood vessels and DAPI (blue) to label nuclei. Bar: 25 μm (i) Representative transmission electron microscopy pictures of high-pressure frozen BAT samples from a SPIO-TRL injected, cold-exposed FVB mouse. L: lipid droplet, M: mitochondrium, C: capillary. Upper panel left, bar: 5 μm; upper panel middle, bar: 1 μm; upper panel right, bar: 0.05 μm; lower panel left, bar: 1 μm; lower panel middle, bar: 0.05 μm; lower panel right, bar: 0.02 μm. *P<0.05; ^{&}P<0.01; ^{$}P<0.001.

FIG. 3. LPL and CD36 drive TRL clearance Into BAT
(a) Organ distribution of ⁵⁹Fe-SPIO and (b) triolein-derived ³H-radioactivity 15 min after intravenous injection in cold FVB mice that were pre-injected with tetrahydrolipstatin (THL) to inhibit LPL activity or with heparin to release LPL into circulation, respectively. Mean values ±s.e.m. with n=5. (e) Oral fat tolerance test in cold FVB mice pre-treated with THL. Mean values ±s.e.m. with n=5. (d) Relative mRNA expression levels in C57BL/6.1 BAT of master genes regulating thermogenesis (Pparg, Ppargc1a, Ucp1, Dio2), lipoprotein binding (Ldlr, Lrp1, Apoe, Gpthbp1, Cd36), lipolysis of lipoproteins (Lpl, Gpihbpl, Angptl4) as well as fatty acid uptake (Fatp1, Fatp3, Fatp4 and Cd36). (e) Determination of Cd36 and other fatty acid transporters mRNA copy numbers by Tat:Man. (f) Consecutive FPLC analysis of TRL-³H-triolein and albumin-³H-oleate in cold-exposed wild-type and Cd36^{-/-60} littermates, (g) Organ distribution of ⁵⁹Fe-SPIO and (h) triolein-derived ³H-radioactivity 15 min after intravenous injection of radiolabeled TRL into Cd36^{-/-} and wild-type littennates. Mean values ±s.e.m. with n≧6. *P<0.05; ^{&}P<0.01; ^{$}P<0.001.

FIG. 4. BAT activation corrects hyperlipidemia and is not impaired in insulin resistance
(a) Triglyceride levels in hyperlipidemic Apoa5^{-/-} mice during cold exposure and (b) pictures of plasma after 24 h cold exposure. (c) Triglyceride and cholesterol FPLC profiling of pooled plasma from Apoa5^{-/-} mice after 4 h and 24 h cold exposure. (d) Environmental scanning electron microscopy studies of brown adipose tissue from lean and obese control and cold-exposed mice. (bar: 25 μm) (e) pictures of interscapular BAT in control and cold-exposed obese mice (bar: 0.5 cm). Combined oral glucose and fat tolerance test in lean and obese control and cold-exposed mice using f, deoxyglucose and (g) ³H-triolein tracers. (h) Turnover kinetics and (i) organ uptake of ³H-triolein-TRL in lean and obese control and cold-exposed mice. Mean values ±s.e.m. with n=6. *P<0.05; ^{&}P<0.01; ^{$}P<0.001.

FIG. 5 SPIO-Nanosomes were injected via the tail vein into a fasted mouse. Due to the increased LPL expression in heart (myocard) upon fasting, there is strong uptake of SPIO-Nanosomes into the myocard. The myocard is enhanced due to the negative contrast caused by SPIO. (a) Transversal T2*-weighted MRI picture before injection of SPIO-Nanosomes. (b) Transversal T2*-weighted MR1 picture after injection of SPIO-Nanosomes. (c) Difference of (a) and (b). The myocard is visualized as an enhanced ring. In a second, equally fasted littermate mouse, 1 min before the SPIO-Nanosomes were injected, LPL was specifically inhibited by injection of a lipase-specific inhibitor (Tetrahydrolipstatin (THL) (200 μl with 1.25 mg/ml THL In PBS with 10% DMSO)). (d) Transversal T2*-weighted MRI-picture before injection of SPIO-Nanosomes. (0) Transversal T2*-weighted MRI-picture after injection of SPIO-Nanosomes. (f) Difference of (e) and (f). The myocard is not enhanced. The blood within the lumen of the heart is enhanced as the SPIO-Nanosomes remain in the circulation because LPL-mediated uptake is blocked.

FIG. 6 SPIO-Nanosomes were injected via the tall vein Into a cold-exposed mouse. Due to the increased LPL expression in brown adipose tissue (BAT) upon cold exposure, there is a strong uptake of SPIO-Nanosomes into the BAT. The BAT is enhanced due to the negative contrast caused by SPIO. (a) Transversal T2*-weighted MRI picture before injection of SPIO-Nanosomes. (b) Transversal T2*-weighted MRI picture after injection of SPIO-Nanosomes. (c) Difference of (a) and (b). The BAT is visualized as five enhanced structures. In a second, equally cold-exposed littermate mouse, 1 min before the SPIO-Nanosomes were injected, LPL was specifically inhibited by injection of a lipase-specific inhibitor (Tetrahydrolipstatin (THL) (200 μl with 1.25 mg/ml THL in PBS with 10% DMSO)). (d) Transversal T2*-weighted MRI picture before injection of SPIO-Nanosomes. (e) Transversal T2*-weighted MRI picture after injection of SPIO-Nanosomes. (f) Difference of (e) and (f). The BAT is not enhanced.

FIG. 7 SPIO-Nanosomes were injected via the tail vein into a cold-exposed mouse. The uptake into the BAT is measured by dynamic MRI. At tr=0 sec, SPIO-Nanosomes were injected. Due to the increased LPL expression in brown adipose tissue (BAT) upon cold exposure, there is strong uptake of SPIO-Nanosomes into the BAT. In a second, equally cold-exposed mouse, 1 min before the SPIO-Nanosomes were injected, LPL was specifically inhibited by injection of a lipase-specific inhibitor (Tetrahydrolipstatin (THL) (200 μl with 1.25 mg/ml THL in PBS with 10% DMSO). In a third, equally cold-exposed mouse, 1 min before the SPIO-Nanosomes were injected, human chylomicrons were injected in a tenfold concentration to saturate specific chylomicron binding sites on the endothelium (100 μl with concentration 100 mg/ml triglycerides). (a) Comparison between cold control mouse (continuous line) and THL-injected mouse (dashed line). (b) Comparison between cold control mouse (continuous line) and chylomicron injected mouse (dashed line).

### EXAMPLES

### Example 1

Brown adipose tissue activity controls triglyceride clearance

### METHODS

Animals and diets. All experimental procedures were performed with approval from the animal care committees responsible for the University Medical Center Hamburg-Eppendorf. Animals were housed at 22° C. with ad libitum access to standard laboratory chow diet. We used male age-matched (16-22 weeks) Ldlr^{-/-}, Apoe^{-/-}, Apoa5^{-/-} and respective FVB wild-type mice as well as LRP1-N2 knockin, Cd36^{-/-} and C57BL/6J wild-type mice which were fasted 4 h prior to the experiment. Control (22° C.) and cold exposure (4° C.) was performed in single cages for 24 h unless indicated otherwise. To induce insulin resistance and obesity, male C57BL/6J mice were single-caged and were at 4 weeks of age fed a diabetogenic high-fat diet³⁰ ad libitum for 16 weeks.

Turnover studies and organ distribution. For turnover studies, anesthetized mice were tail vein injected with 200 μl radiolabeled TRL. Lipoprotein turnover was determined from 10 μl plasma 0.5, 1, 2, 5 and 15 min after injection. After 15 min, blood was removed by cardiac puncture, the right atrium was opened, and the carcass was perfused through the left ventricle with PBS containing 50 U mL¹ heparin. Then, organs were harvested and weighed. For measurement of radioactivity, organs were solubilized in Solvable (PerkinElmer, Boston, USA, 0.1 mL per 10 mg organ), 200 μL were counted in scintillation fluid and TRL uptake was calculated as c.p.m. per mg organ. Oral fat tolerance tests were performed by gavage of 100 μL olive oil with [9,10-³H(N)]-triolein (370 KBq per mouse). To measure lipoprotein production triton WR-1339 (Tyloxapol from Sigma; 0.5 mg per g body weight as 10% solution in PBS) was injected into the tail vein. Plasma was collected at indicated time points. For the measurement of hepatic production 1(3)-³H glycerol (125 KBq per mouse) was injected prior to triton WR-1339. Lipids were extracted from plasma samples and ³H-glycerol incorporated into triglycerides was measured as described above. For chylomicron production mice received ³H-triolein in olive oil by gavage as described above directly after triton WR-1339 injection. Intestine-derived radioactivity in plasma was measured as described above. For manipulation of LPL function, THL (Roche, 12.5 mg ml⁻¹ DMSO) was diluted to 1.25 mg ml⁻¹ in 10% DMSO in PBS. Mice received 200 μL of either 0.25 mg THL, 50 U heparin (ratiopharm) or 10% DMSO in PBS (mock). After 1 min ⁵⁹Fe-SPIO- and ³H-triolein-labeled TRL were injected and plasma clearance and organ uptake were determined as described above. For postprandial studies mice were i.p. injected with 0.25 mg THL or mock solution prior to gavage of 200 μL olive oil. Blood was collected at indicated time points and plasma triglyceride levels were determined.

In vivo imaging studies. MRI was performed as described before. Briefly, all static and dynamic MRI measurements were performed with a clinical 3 Tesla MR scanner (Philips Medical Systems, Netherlands) equipped with a custom-made small animal solenoid coil. The dynamic measurements were based on a gradient-echo sequence (Supplementary table 3). The applied sequence is highly sensitive to susceptibility effects caused by local magnetic field inhomogeneities caused by SPIO-TRL. DICOM data were processed with Image (http://rsbweb.nih.gov/ij/). For cryo electron microscopy, SPIO-TRL were intravenously injected into control or cold wild-type FVB mice. After 30 min mice were sacrificed, BAT biopsies were taken and processed for transmission electron microscopy (TEM) as described. Micrographs were obtained with a FBI Eagle 4k CCD camera and a Technal 20 TEM operated at 200 kV. For environmental scanning electron microscopy studies of BAT anaesthetized mice were perfused with PBS-Heparin as above and organs were fixed with 2.5% glutaraldehyde in PBS, washed, and postfixed for 30 min with 1% Osaᵢ in PBS. For intravital microscopy interscapular BAT was dissected in anaesthetized mice and visualized by a confocal microscope equipped with a resonant scanner (Nikon AIR). QD-labeled TRL and fluorescent probes were injected via a tail vein catheter and 15 or 30 confocal images per second were recorded. The acquired data sets were aligned to reduce object movements due to mouse breathing and denoised with a Savitzky-Golay filter in Nikon NIS Elements AR 3.10. Labeling, animations and quicktime-export were done with Adobe After Effects CS4.

Statistics. To assess statistical significance two-tailed, unpaired Student's t-test or two-way ANOVA followed by post-hoc Bonferroni's test was performed. P<0.05 was considered significant.

Preparation and labeling of TRL, plasma parameters, RNA extraction and real-time quantitative PCR, endothelial permeability testing and Western blotting were performed as known in the art. Preparation of TRL comprising SPIO was performed essentially as disclosed in Bruns et al. Nature Nanotechnology 4, 2008:193-201 and the supplement to said publication(¹⁴), with the modification that nanocrystals with a size of about 10 nm were used, The increased size led to a better signal.

### RESULTS

Brown adipose tissue (BAT) burns fatty acids for heat production in order to defend the body against cold^{1,2} and has recently been shown to be present in humans³⁻⁵. Triglyceride-rich lipoproteins (TRL) transport lipids in the bloodstream, where fatty acids are liberated by the action of lipoprotein lipase (LPL)⁶. Fatty acids are taken up by peripheral organs such as muscle and adipose tissue, whereas remaining cholesterol-rich remnant particles are cleared by the liver⁶. Elevated triglycerides and prolonged circulation of remnants, especially in diabetic dyslipidemia, are risk factors for cardiovascular disease⁷⁻¹¹. However, the precise biological importance of BAT for TRL clearance remains unclear. Here, the inventors show that increased BAT activity induced by short-term cold exposure controls TRL metabolism in mice. Cold exposure drastically accelerated plasma clearance of triglycerides as a result of increased uptake into BAT, a process crucially dependent on local LPL activity and transmembrane receptor CD36. In pathophysiological settings, cold exposure corrected hyperlipidemia and improved deleterious effects of insulin resistance. In conclusion, BAT activity controls vascular lipoprotein homeostasis by inducing a metabolic program that boosts TRL turnover and channels lipids into BAT. Activation of BAT might be a therapeutic approach to reduce elevated triglyceride levels and combat obesity in humans.

To determine whether cold exposure alters the lipoprotein profile, plasma from FVB wild-type mice kept at 22° C. (control mice) or at 4° C. in a cold room (cold mice) was analyzed by fast performance liquid chromatography (FPLC). TRL-triglycerides were markedly reduced after 4 h and 24 h (FIG. 1a) demonstrating that cold exposure lowers triglyceride levels efficiently. HDL-cholesterol, however, is slightly increased (FIG. 1b) which is probably explained by an increase of TRL-derived HDL precursors¹². After a fatty meal, triglyceride-rich chylomicrons transport dietary lipids, but it is unclear whether BAT is involved in their processing. Therefore an oral fat tolerance test was performed with olive oil mixed with ³H-triolein in wild-type FVB mice (FIG. 1c). In control mice, triglyceride levels rose with a peak after 2 h and decline as expected^{6,13}. In cold mice, triglyceride levels remained persistently low during the postprandial phase. The lipoprotein profile (FIG. 1d and Supplementary FIG. 1) confirmed the presence of chylomicrons in control mice whereas they were absent in cold mice. The corresponding ³H-radioactivity profile resembled the curve for triglycerides in control mice, while in cold mice plasma ³H-radioactivity steadily rose (Supplementary FIG. 2).

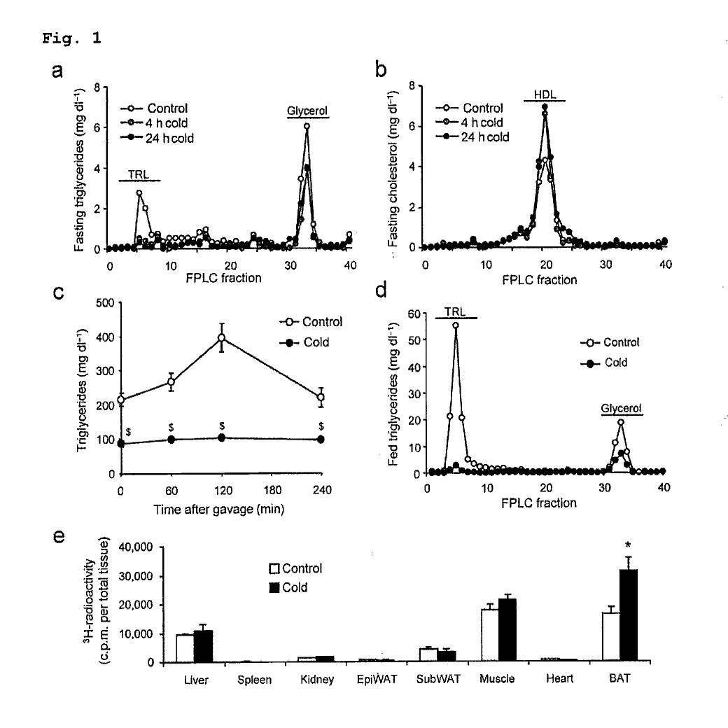

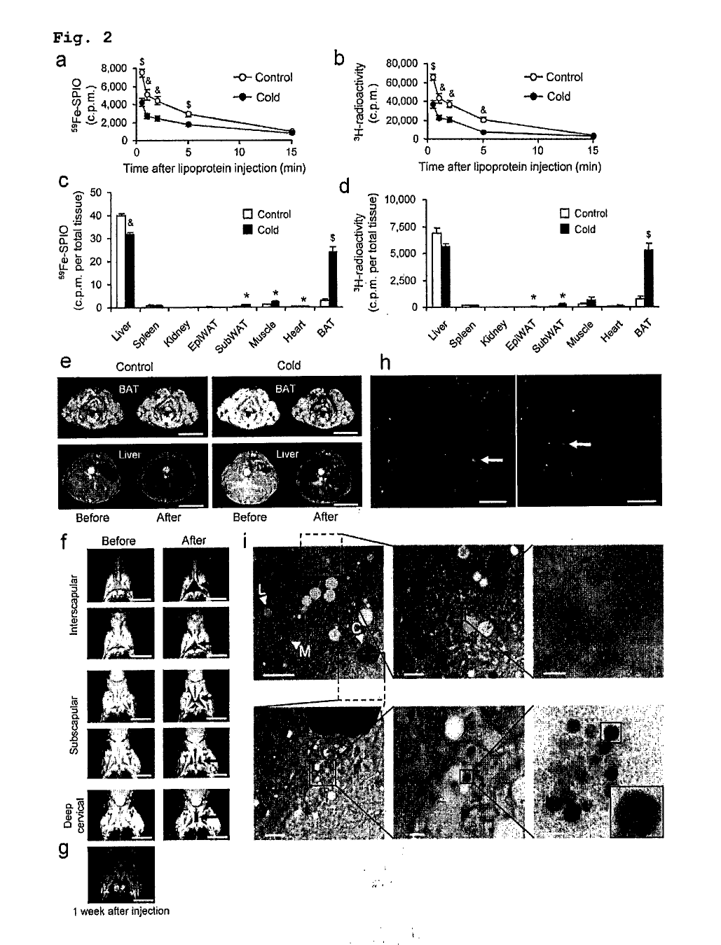

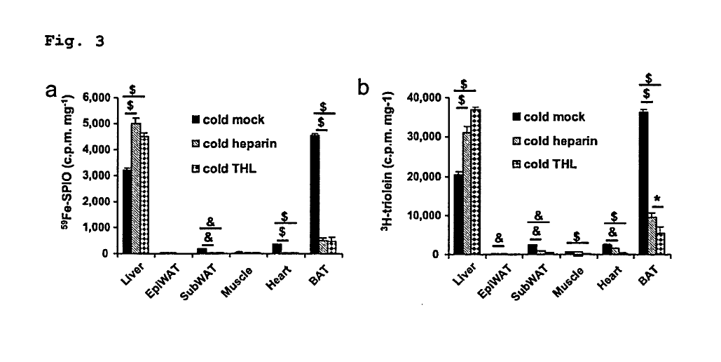

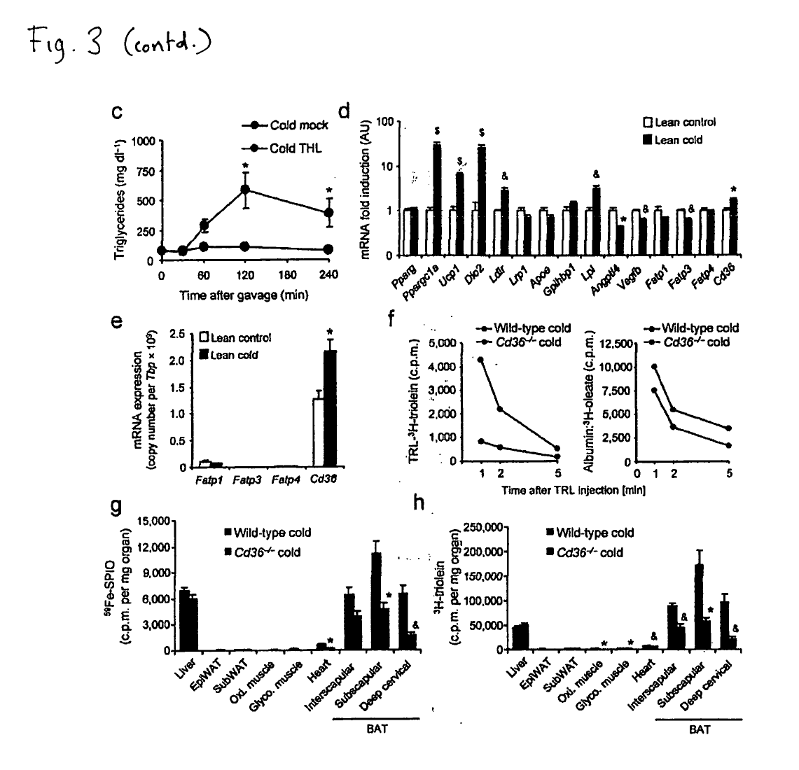

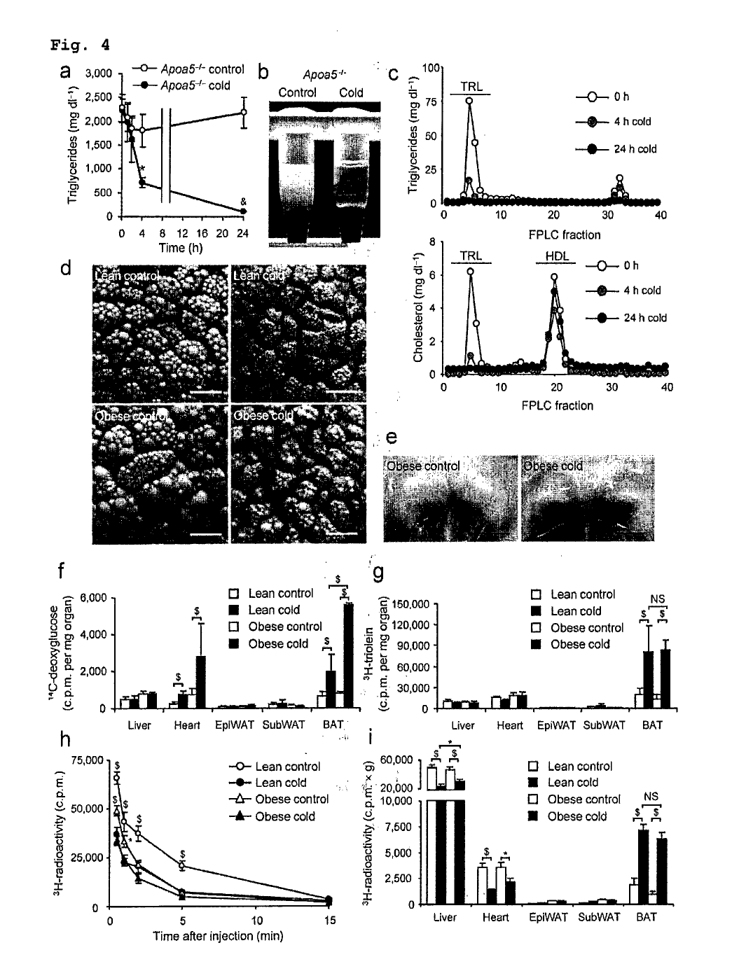

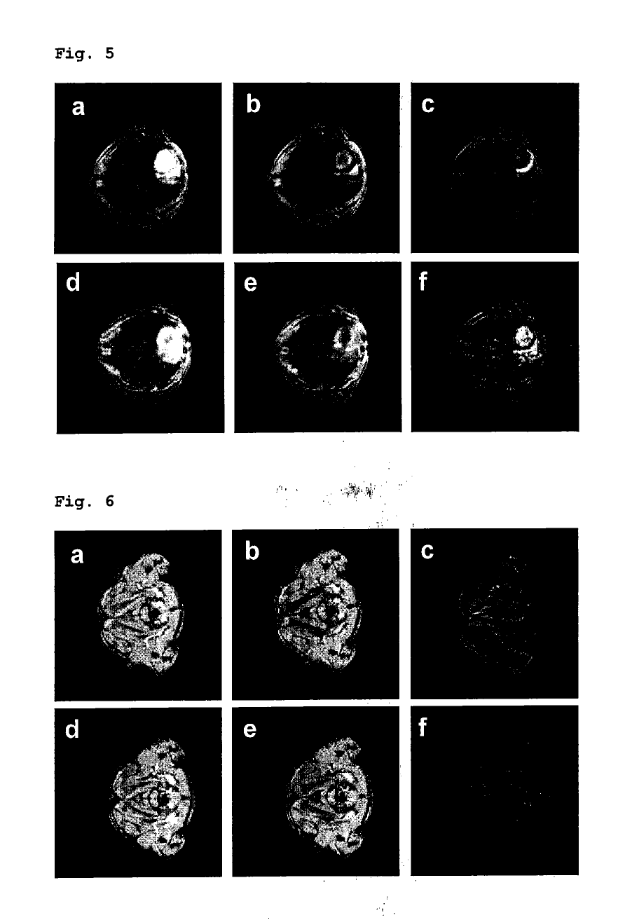

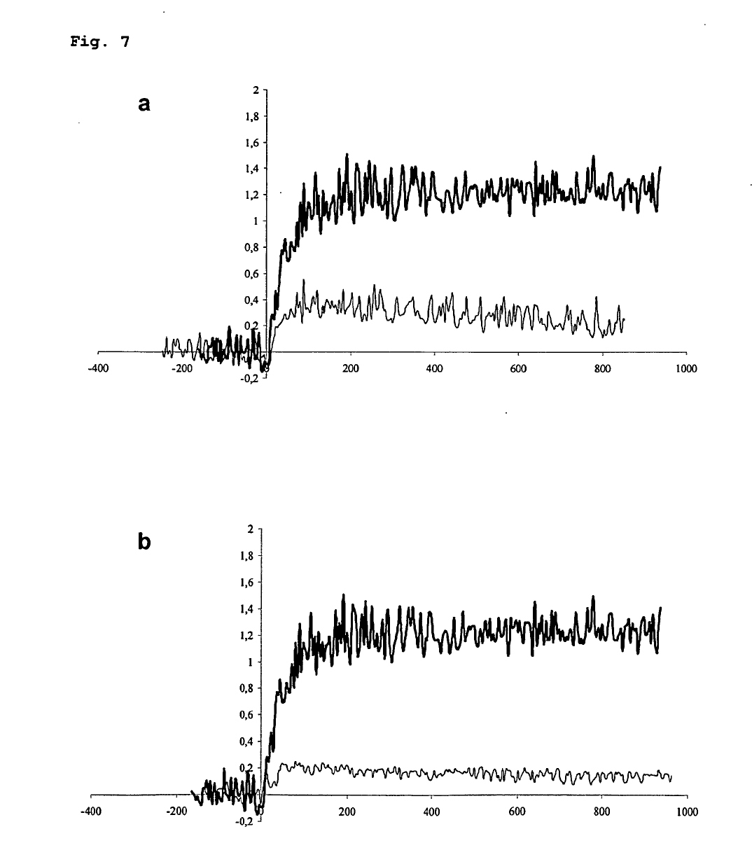

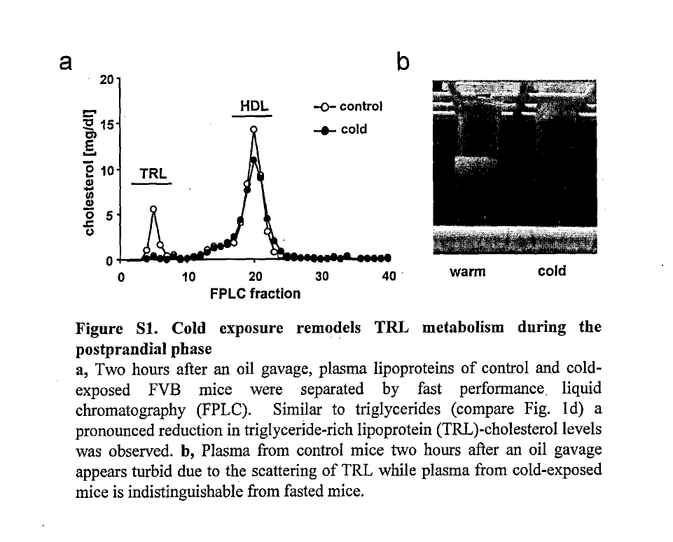

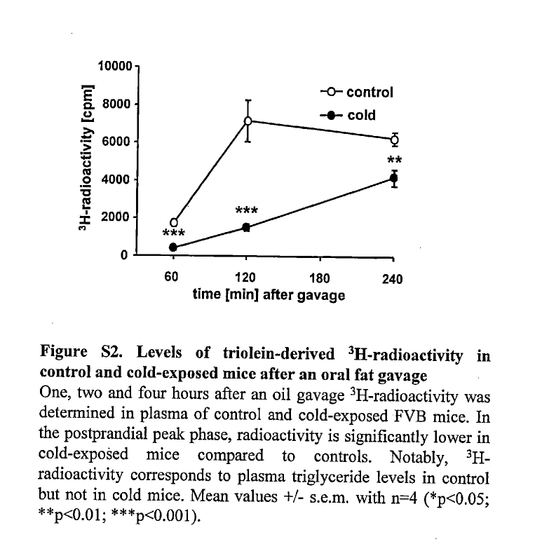

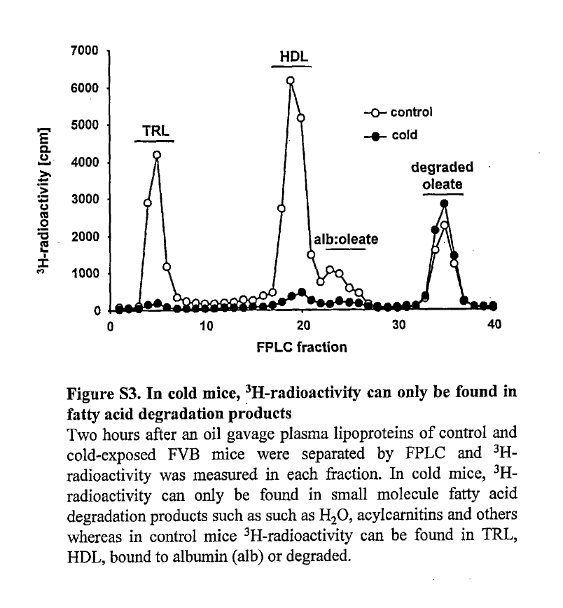

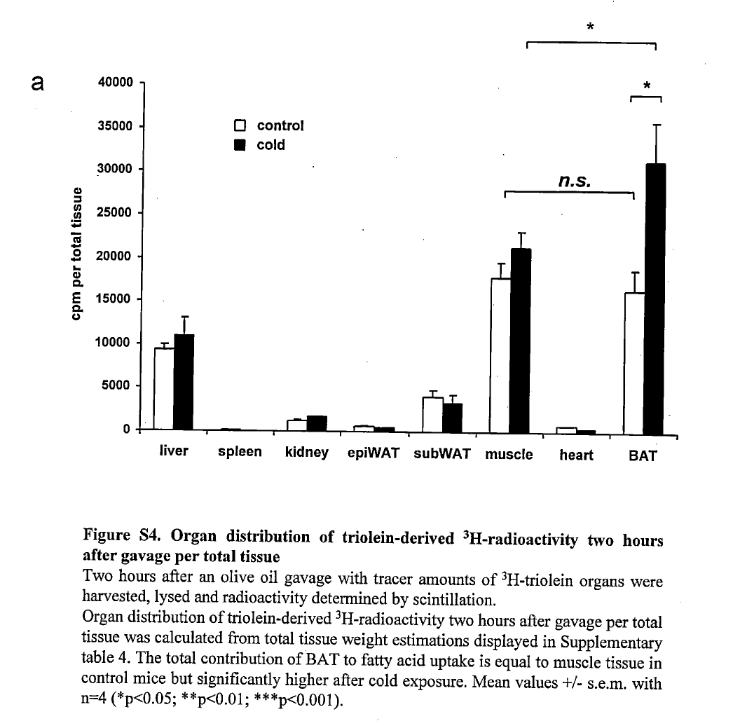

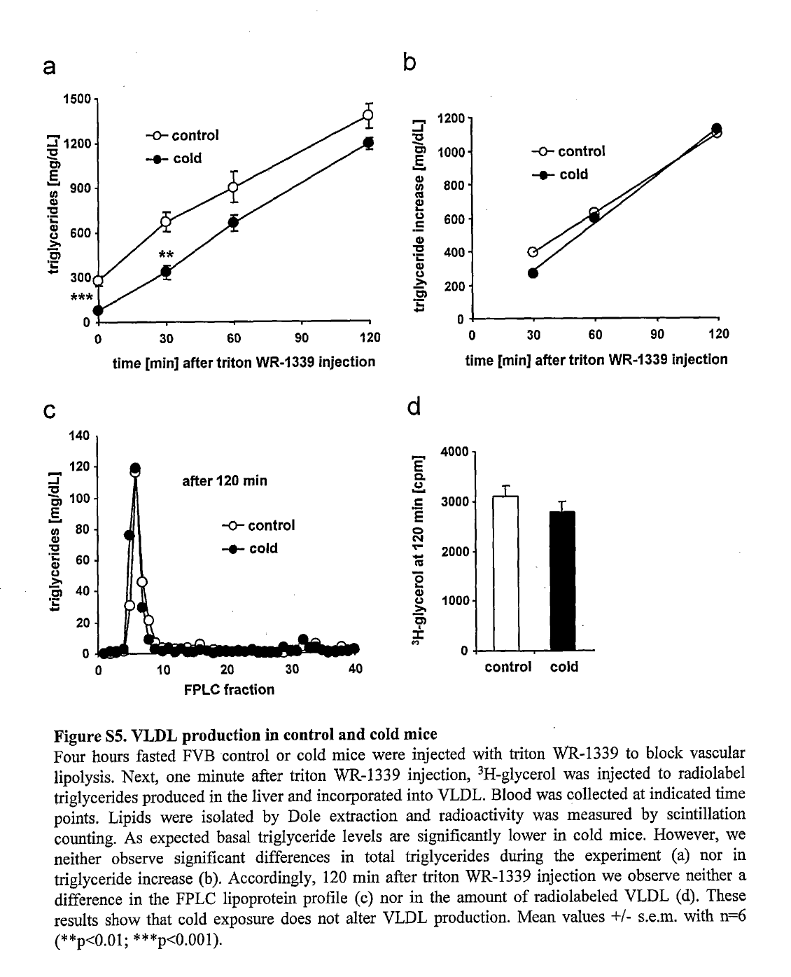

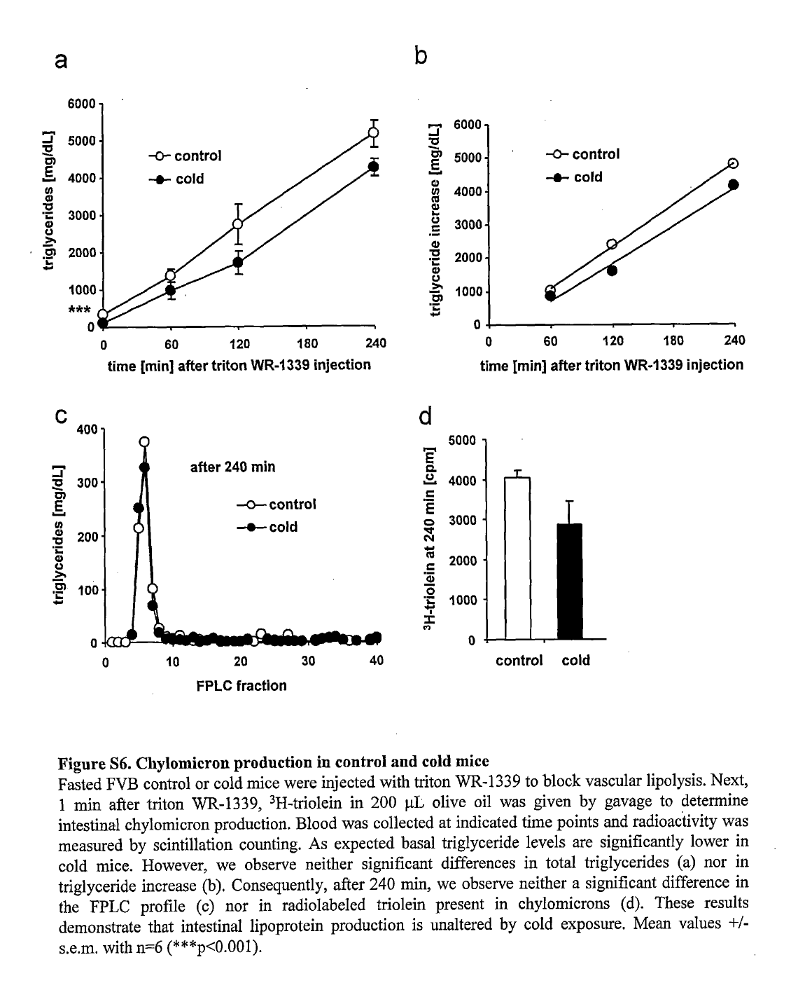

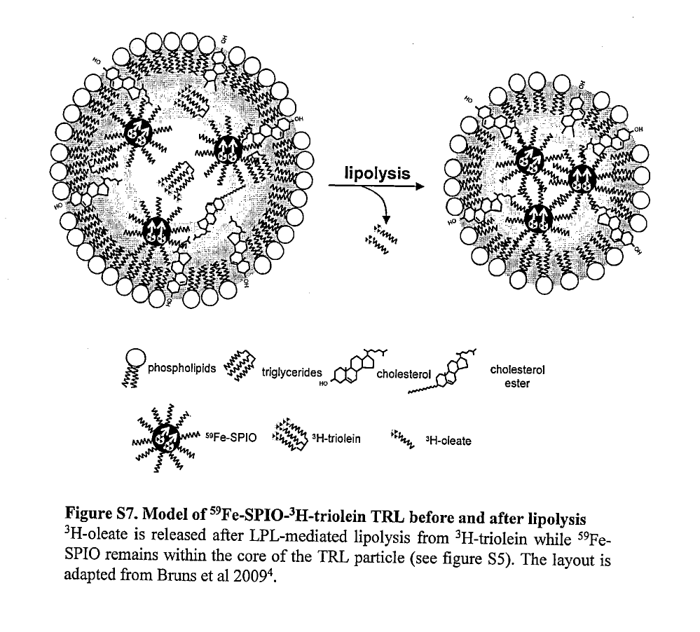

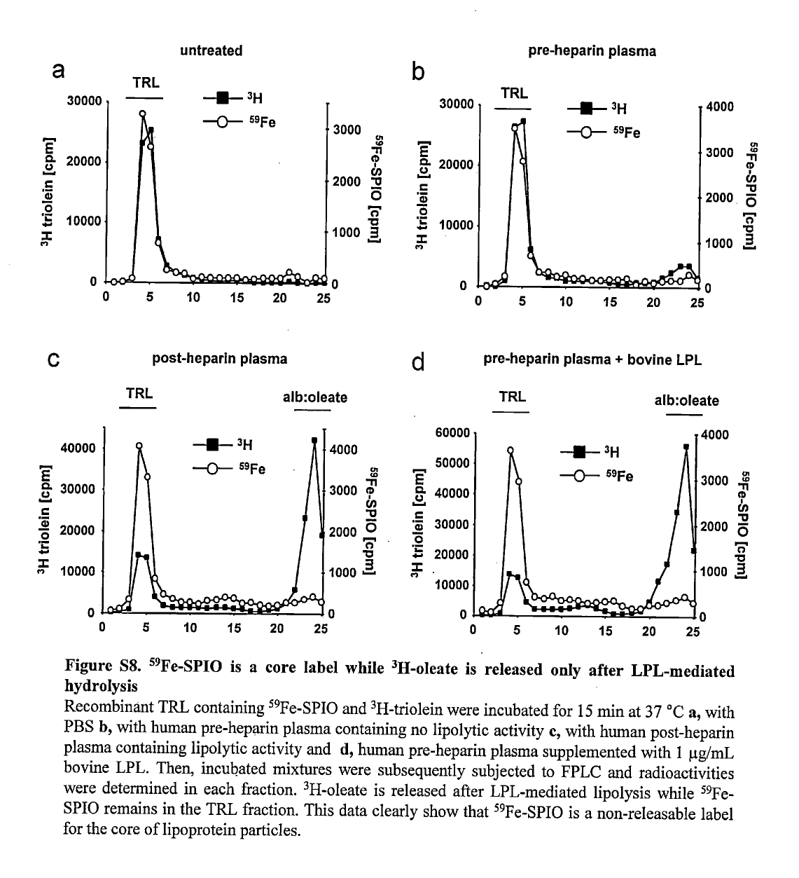

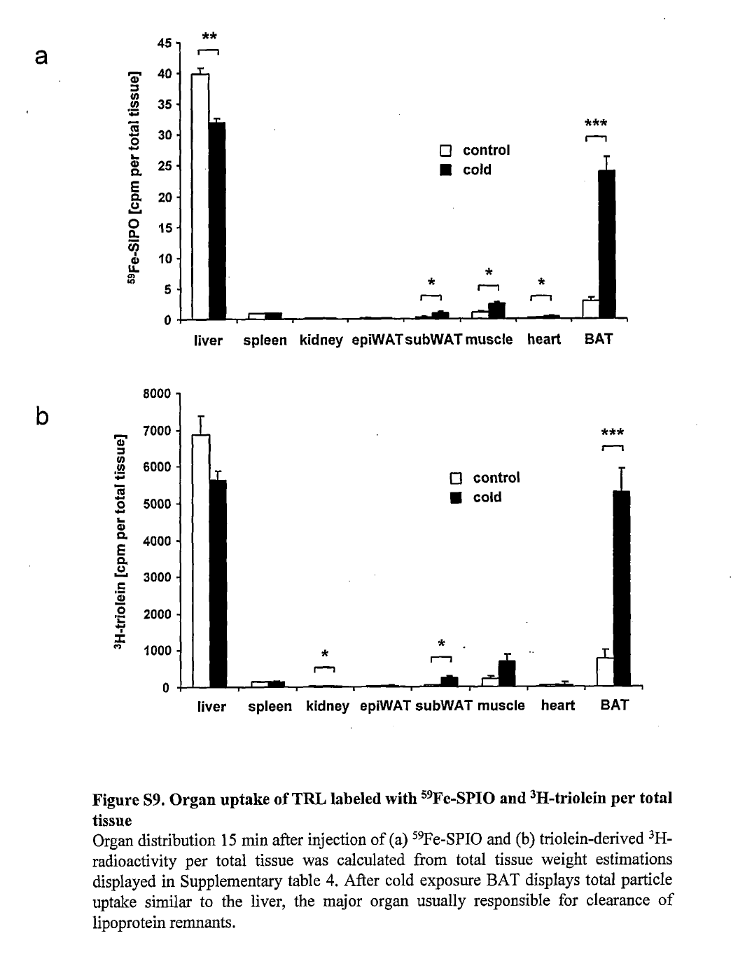

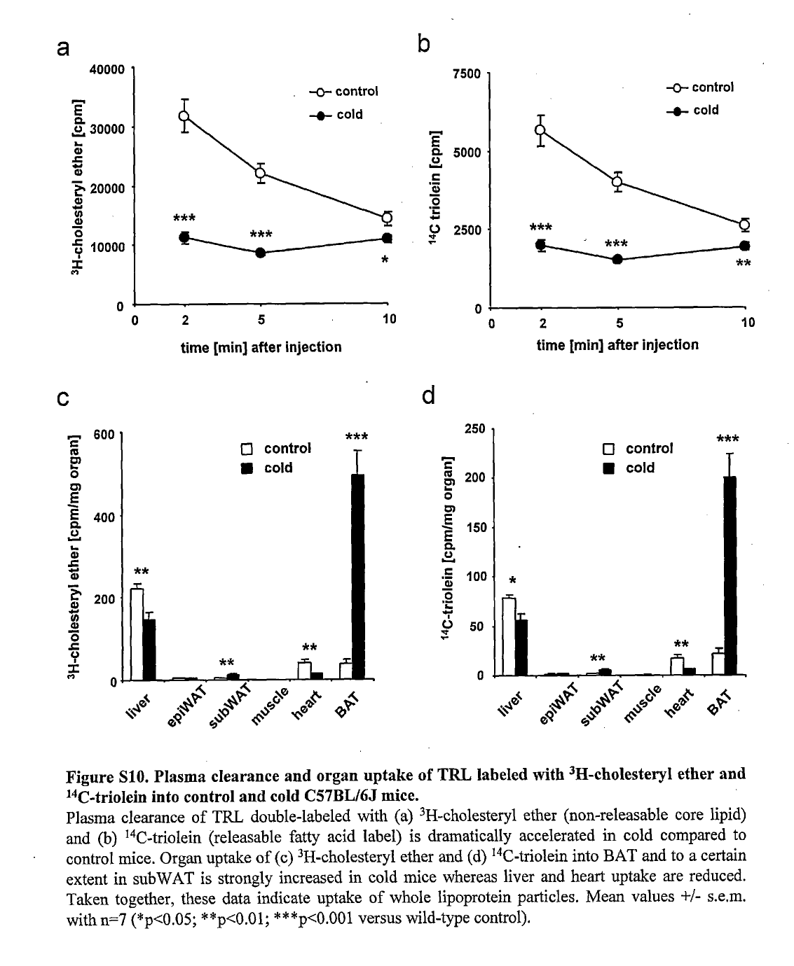

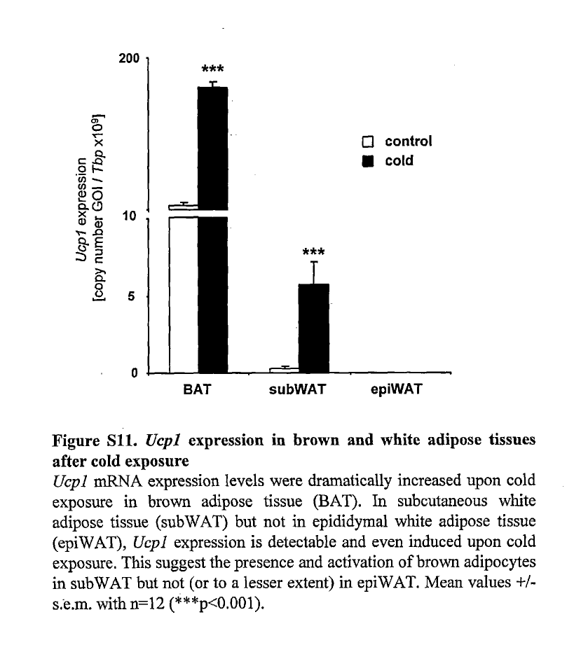

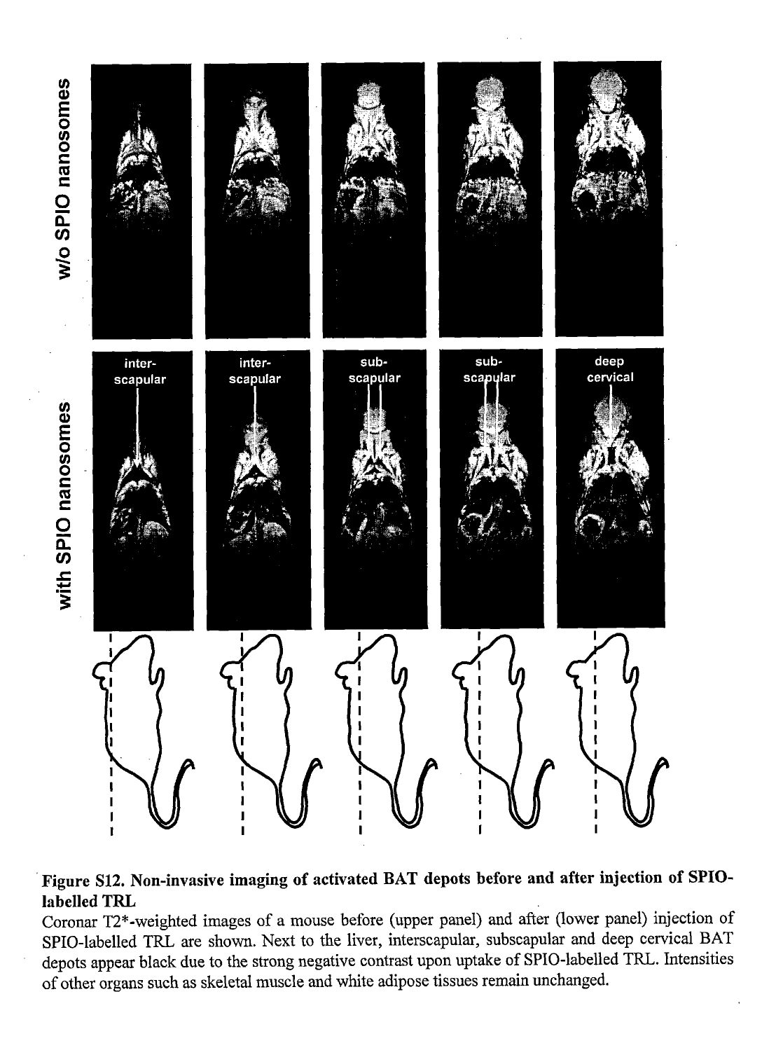

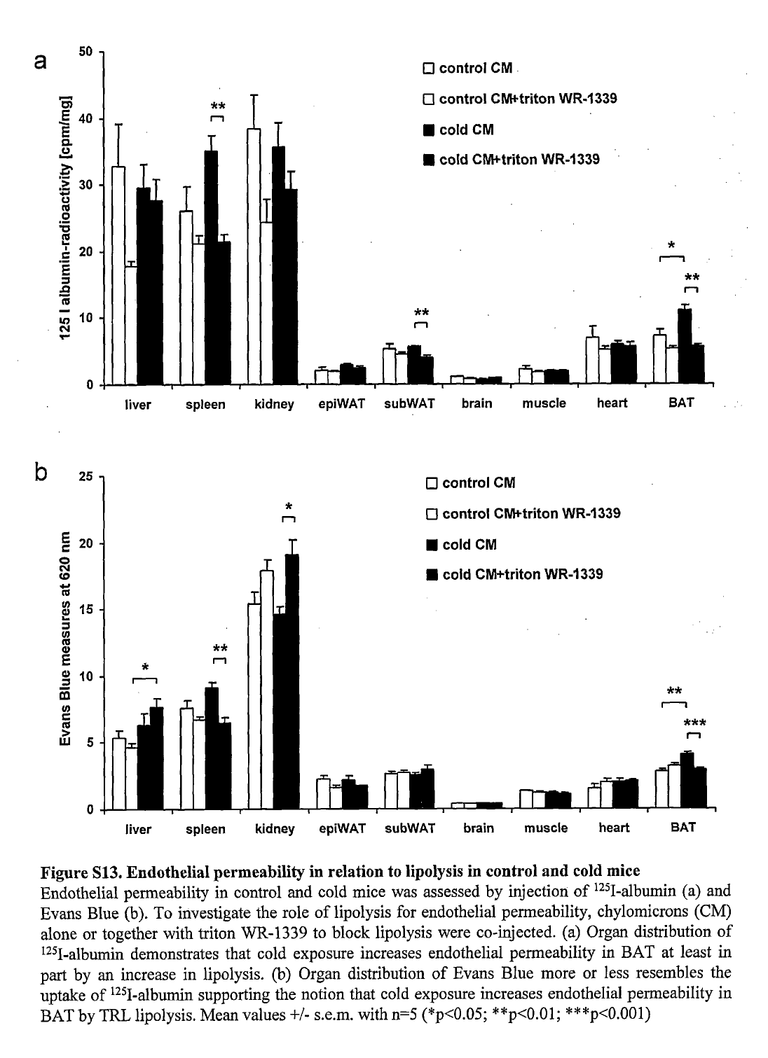

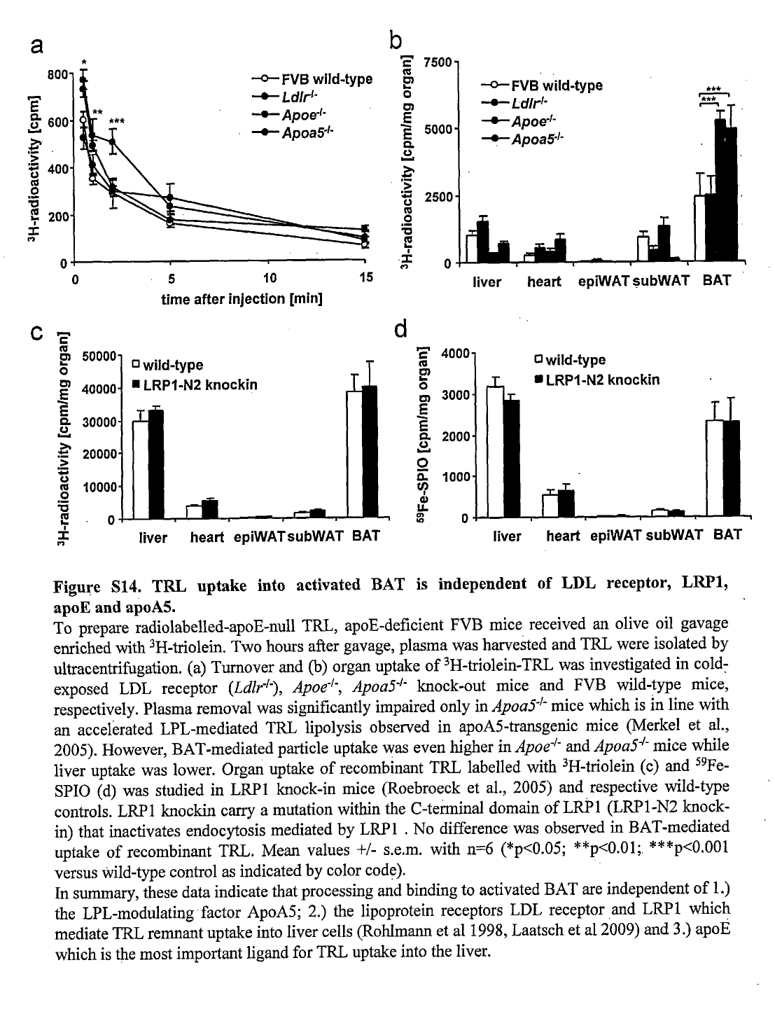

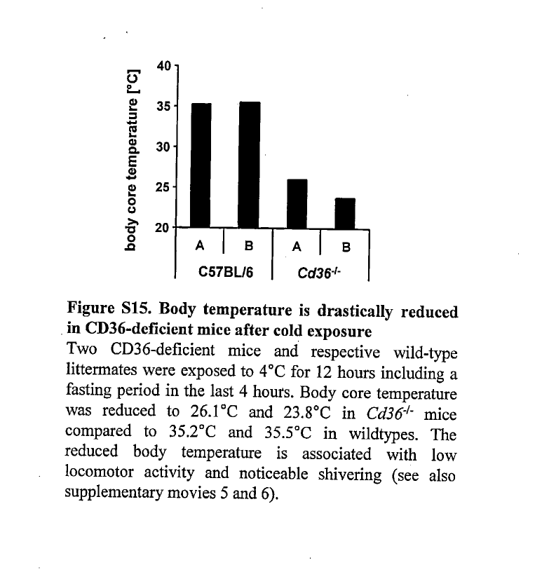

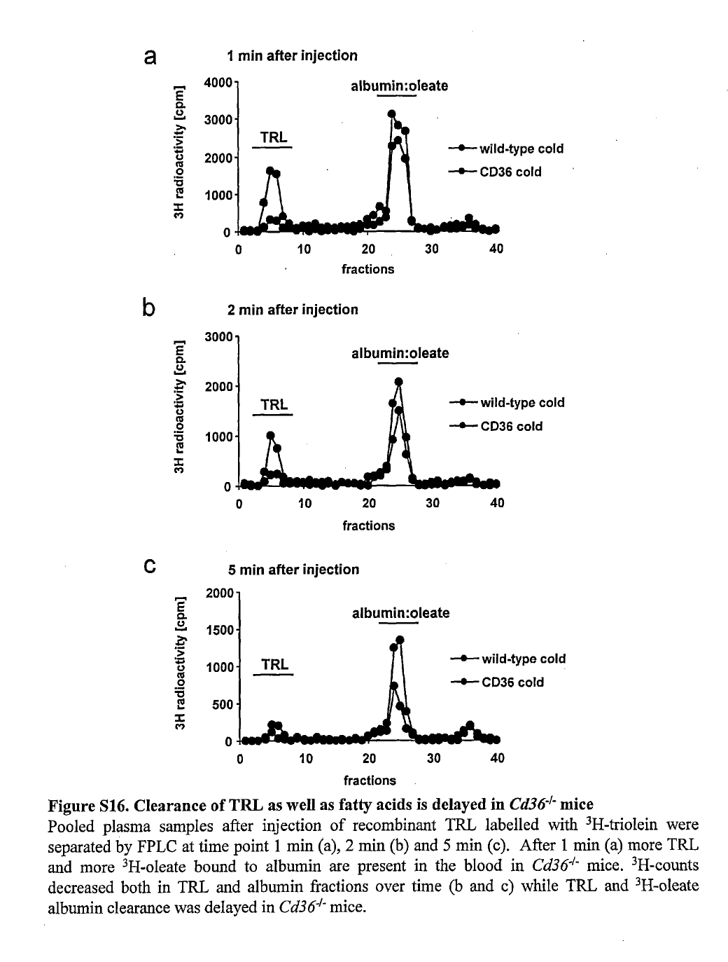

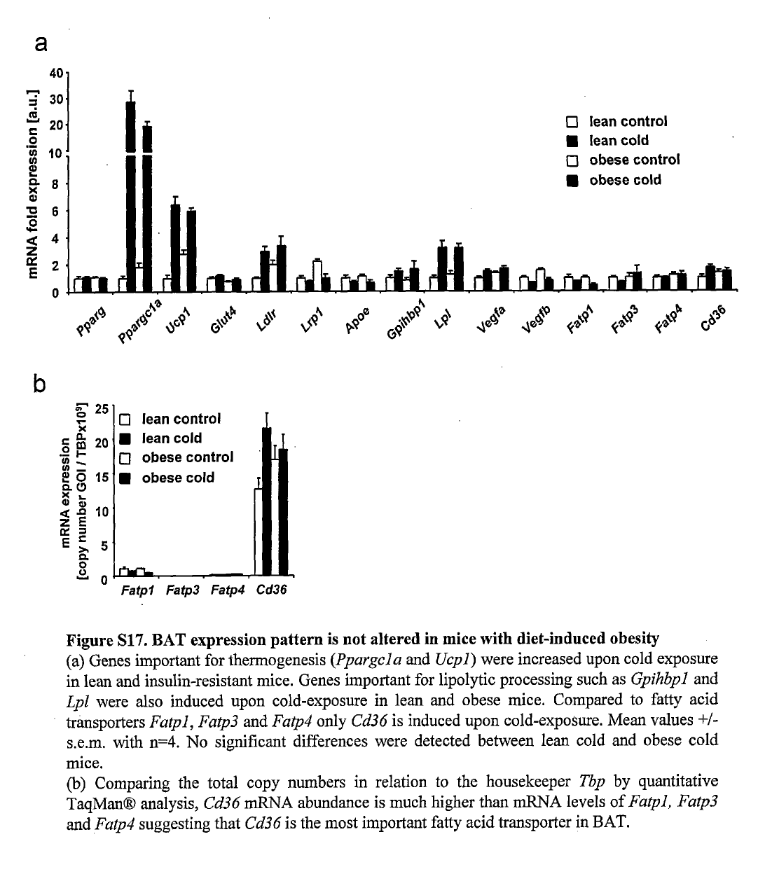

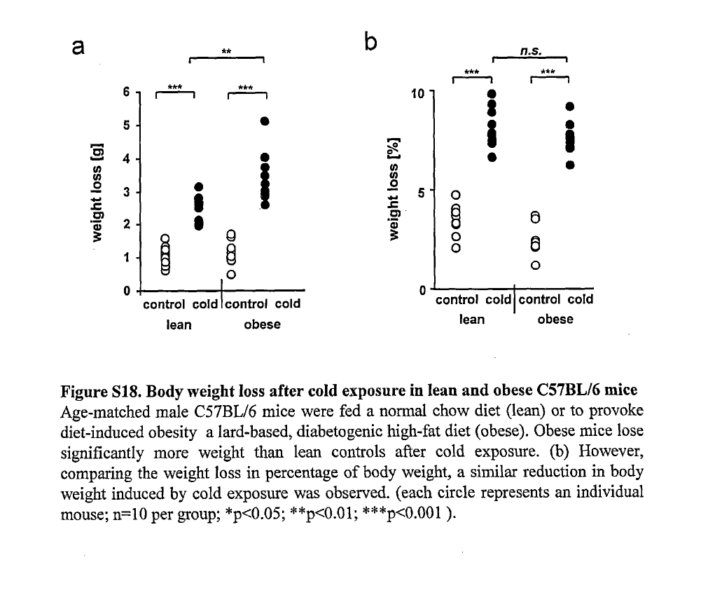

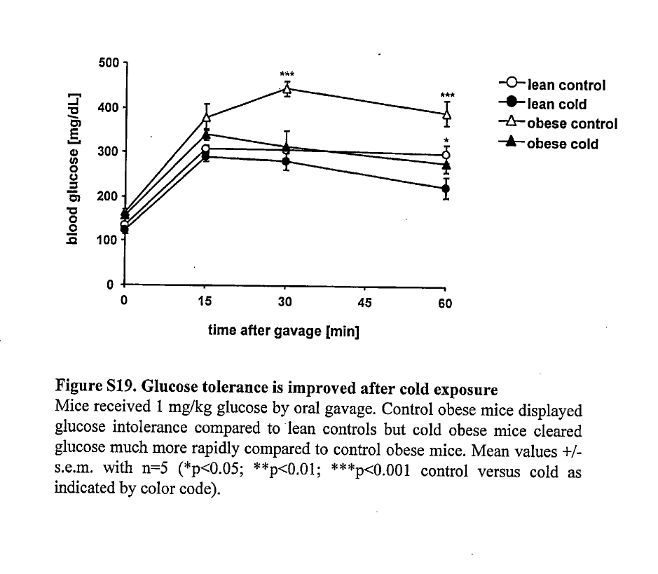

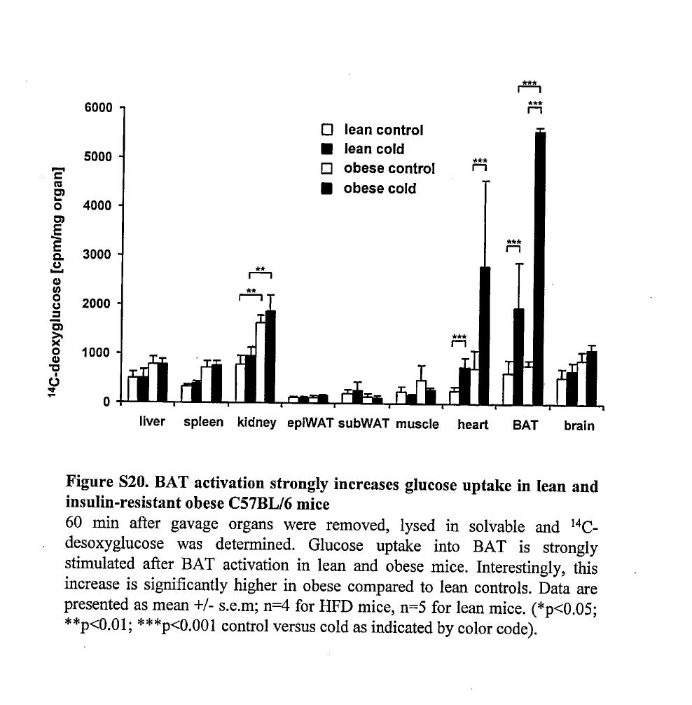

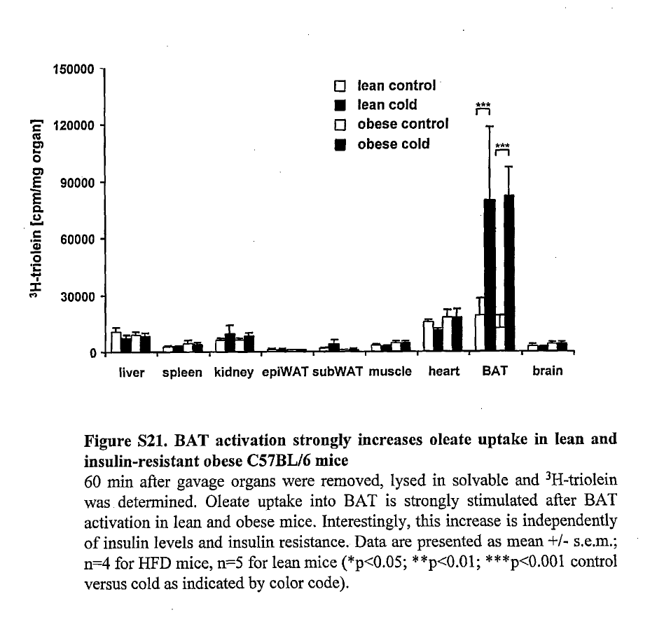

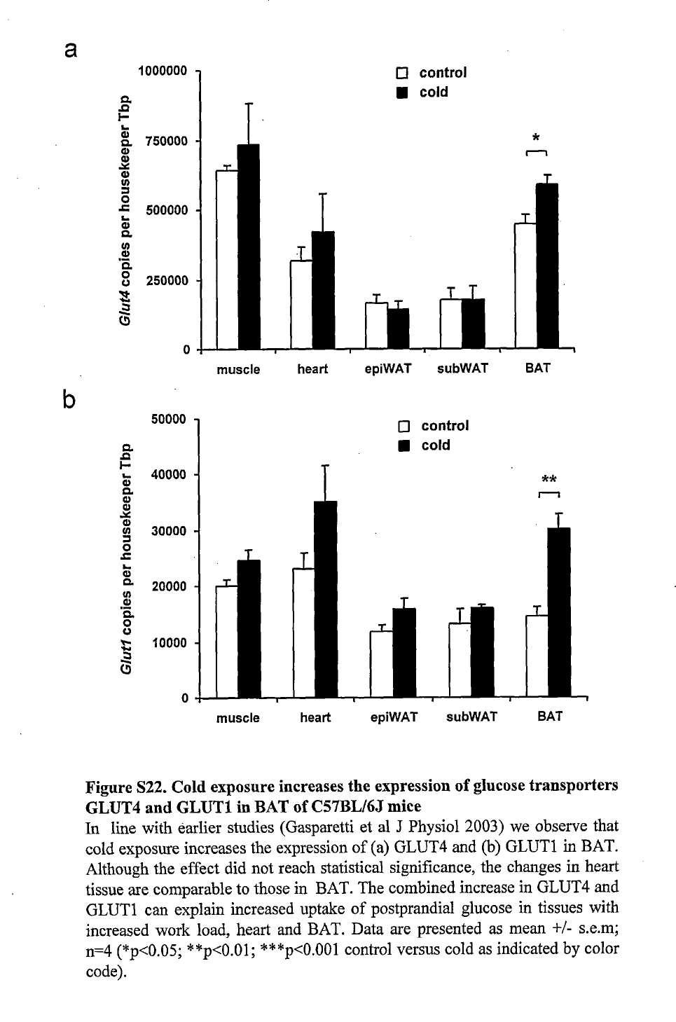

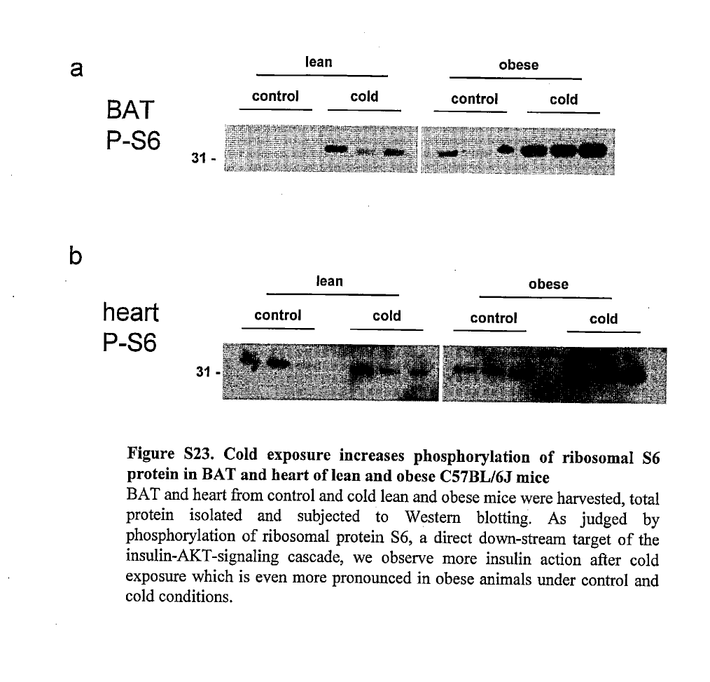

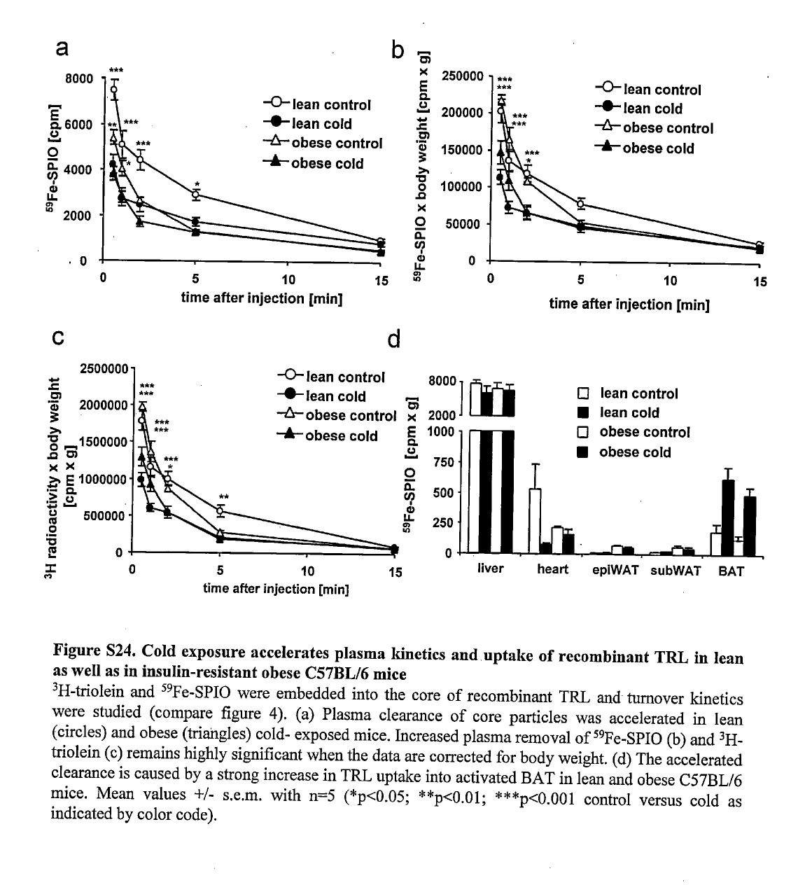

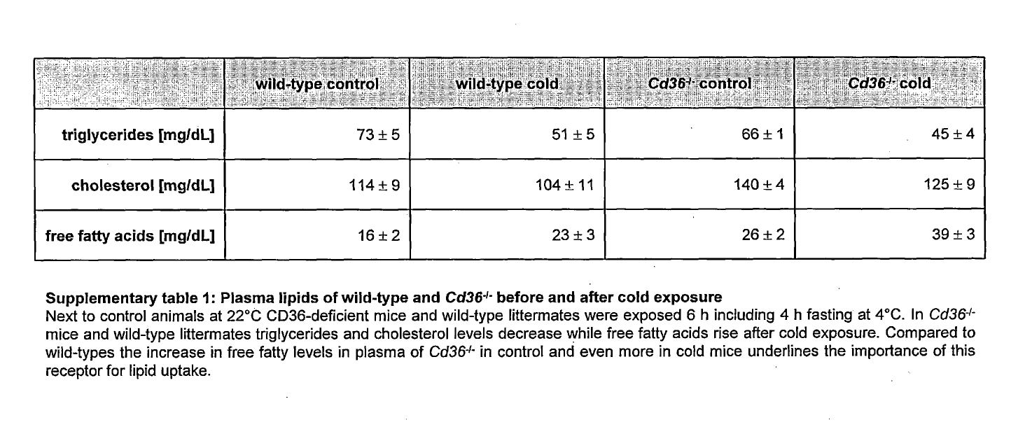

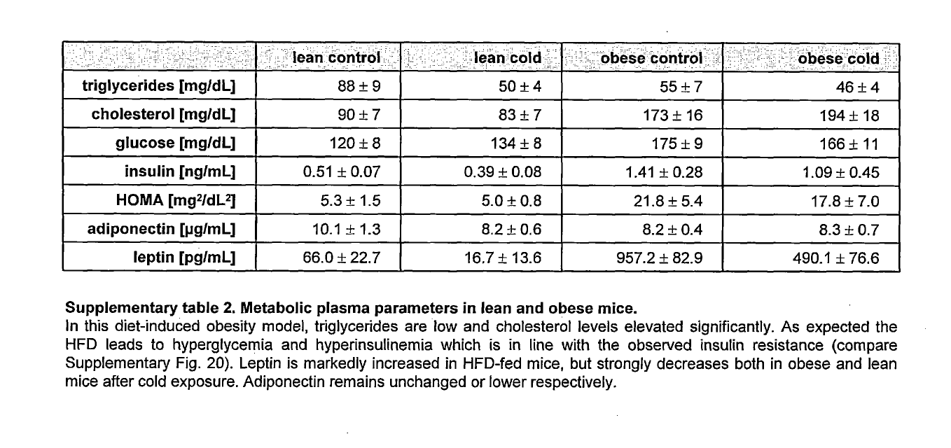

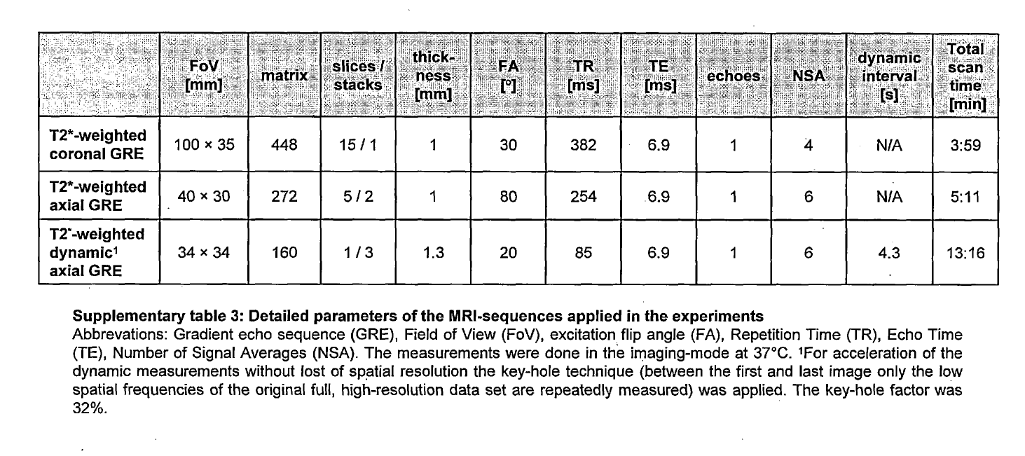

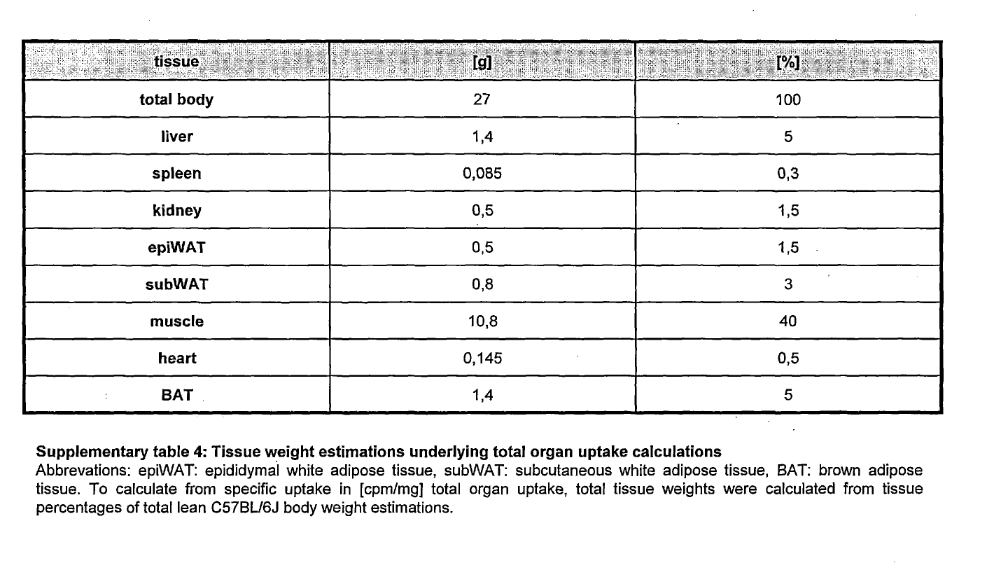

The latter can be explained by the occurrence of small molecule fatty acid degradation products in the blood (Supplementary FIG. 3). Organ distribution of ³H-triolein-derived radioactivity 2 h after gavage (FIG. 1e and Supplementary FIG. 4) revealed a selective increase in organ uptake of fatty acids into BAT. The total contribution of BAT was higher than for muscle which also participates In heat production In response to acute cold by shivering thermogenesis¹. As the production rates of hepatic (Supplementary FIG. 5) as well as of intestinal TRL (Supplementary Fig: 6) were unaltered by cold exposure, the increase in specific uptake of TRL-lipids into different BAT depots suggests an accelerated clearance of postprandial TRL in cold mice. To investigate the clearance and kinetics in more detail, both ³H-triolein and hydrophobic ⁵⁹Fe-superparamagnetic iron oxide (SPIO) nanocrystals were embedded into the core of TRL particles. These particles allowed to following lipoprotein and fatty acid uptake simultaneously, because upon LPL-mediated lipolysis ³H-oleate was released, while hydrophobic ⁵⁹Fe-SPIO nanocrystals remained within the TRL core (Supplementary FIGS. 7 and 8). Clearance of both TRL-derived ⁵⁹Fe-SPIO (FIG. 2a) and ³H-triolein (FIG. 2b) was significantly faster in cold compared to control mice. The organ distributions of ³H-triolein and ⁵⁹Fe-SPIO indicated that the accelerated clearance was mediated by an approximately 10-fold increase in specific uptake into BAT (FIG. 2c,d and Supplementary FIG. 9). Total amounts of ³H-triolein and “Fe-SPIO uptake in BAT were comparable to total liver uptake while the contribution of other tissues was small. We confirmed these findings using non-hydrolysable ³H-cholesterol ethers, a conventional TRL core label (Supplementary FIGS. 10 and 11). The concomitant reduced hepatic TRL uptake indicated that cold exposure shifted the clearance of lipoproteins from liver to BAT. Notably, uptake into subcutaneous white adipose tissue was also increased which can be explained by the presence and activation of brown adipocytes after cold exposure (Supplementary FIG. 12). Recently, hydrophobic SPIO nanocrystals that accelerate spin-spin relaxations were embedded into the TRL core to follow lipoprotein uptake into the liver by dynamic magnetic resonance imaging (MRI)¹⁴. Irrespectively of BAT activity, we observed uptake into the liver of control and cold-exposed mice (FIG. 2e). However, cold exposure markedly increased the negative contrast of several BAT depots, indicative for increased TRL presence (FIG. 2e,f and Supplementary FIG. 13), We observed a pronounced negative contrast in BAT even one week after injection (FIG. 2g) suggesting uptake of the entire SPIO-labeled lipoprotein particle. Intravital microscopy enables to study physiologic processes in vivo on a cellular level. We visualized the vascular circulation and structure of interscapular BAT in real time. In cold-exposed mice, BAT-mediated processing of TRL labeled with hydrophobic fluorescent nanocrystals (QD-TRL) revealed a rapid attachment to the endothelium which was followed by QD-TRL internalization (FIG. 2h). Cryo electron microscopy studies showed that in cold mice, SPIOs were detected underneath capillaries of BAT 30 min after injection indicating TRL particle internalization (FIG. 2i). It has been shown that TRL lipolysis products cause a decrease In endothelial barrier function”. By injection of Evans Blue or ¹²⁵I-labeled albumin with or without inhibiting lipolysis; we found that endothelial permeability in BAT is increased upon cold exposure and that this process was dependent on simultaneous lipolysis (Supplementary FIG. 14). These findings indicate that cold exposure-induced increase in lipoprotein turnover remodels endothelial permeability, thereby allowing an increased internalization of TRL into BAT. Taken together, activated BAT accelerates plasma TRL turnover and is a major target organ for TRL uptake.

To gain further mechanistic insight into BAT-mediated TRL processing, we studied turnover and organ uptake of radiolabeled TRL in mouse models that display defective function of proteins important for lipolysis (apoAV)^{13,16} and particle uptake (apoE, LDL receptor, LRP1)¹⁷⁻²¹, but none of them displayed a reduced uptake into BAT (Supplementary FIG. 15); moreover, uptake was increased in apoE- and apoAV-deficient mice probably due to impaired liver uptake.

To assess whether the canonical LPL pathway is involved in uptake of TRL Into BAT, we Inhibited LPL activity by injecting tetrahydrolipstatin (THL), a specific inhibitor²². Local LPL activity in BAT is required for the uptake of TRL, as THL pre-treatment abolished uptake of both ⁵⁹Fe-SPIO and ³H-triolein into BAT of cold mice (FIG. 3a,b). Uptake into the heart was also inhibited. The results show that uptake of the TRL (the nanosomes) is dependent on LPL activity.

In addition, the inventors showed that release of LPL from the endothelium by heparin pre-treatment also blocked uptake of ³H-triolein and ⁵⁹Fe-SPIO into BAT. It is noteworthy that heparin leads to transient maximized LPL activity in the blood stream²³, however, the amount of fatty acids internalized into BAT under these conditions was very low compared to mock-treated mice. These results indicate that local LPL activity in BAT drives lipolysis and is required for fatty acid as well as for TRL particle uptake into BAT (FIG. 3a,b). In line with FIG. 1c, mock-treated cold mice showed no increase in plasma triglycerides after lipid gavage. In contrast, THL-treated cold animals displayed a significant postprandial triglyceride response supporting a role of LPL for triglyceride clearance in cold mice (FIG. 3c). Intravital confocal imaging depicted that after initial TRL binding to the vascular wail, fluorescent-labeled TRL can be released by heparin. However, time-delayed heparin injection had no influence on already internalized TRL particles, but blocked binding of a second bolus of TRL. Taken together, uptake of TRL into BAT comprises heparin-sensible initial binding to the vessel wall and subsequent internalization of particles in a LPL-dependent manner.

To find candidates that could influence TRL or fatty acid uptake, we analyzed the gene expression profile of BAT from C57BL/65 mice after cold exposure using real-time PCR. (FIG. 3d). Among regulated genes some are known factors for thermogenesis (Ppargc1a, Ucp1, Dio2)¹, some are involved in lipoprotein metabolism (Apoe, Lrp1, Lpl, Gpihbpl, Angptl4, Ldlr, Cd36)^{6,24-27} and some are important for fatty acid uptake (Fatp1, Fatp3, Fatp4 and Cd36)^{28,29}. The expression of the gene coding for the adipocyte master transcription factor peroxisome proliferator-activated receptor y (Pparg) was not influenced. VEGF-B has recently been described to facilitate endothelial fatty acid uptake by a specific stimulatory effect on Fatp3 and Fatp4 expression²⁸. The observation that expression levels of Cd36, a gene coding for a transmembrane lipid and lipoprotein receptor, were significantly increased and the highest absolute whereas other fatty acid transporters and Vegfb had a rather decreased expression (FIG. 3e), prompted us to analyze TRL metabolism after cold exposure in Cd36^{-/-} mice. The crucial importance of CD36 was conceivable, because approximately 60% of the Cd36 mice died during the 24 h cold exposure. Therefore, the exposure time was reduced to 12 h leading to a drastically reduced body temperature in Cd36 mice (Supplementary FIG. 16) associated with low locomotor activity and noticeable shivering (Supplementary movies 6 and 7). After 12 h recovery at room temperature, Cd36^{-/-} mice were indistinguishable from wild-type. The increase in free fatty acid levels in plasma of Cd36 mice which was even more pronounced after cold exposure underlines the importance of this receptor for lipid uptake (Supplementary table 1). FPLC analyses demonstrated that this phenotype correlated not only with a slower turnover of ³H-triolein-TRL but also clearance of ³H-oleate bound to albumin was delayed compared to cold wild-types (FIG. 3f and Supplementary FIG. 17). Consequently, we observed a significant reduction in ⁵⁹Fe-SPIO- as well as ³H-triolein-TRL (FIG. 3g,h) uptake into BAT, demonstrating that CD36 is important for both fatty acid and lipoprotein particle uptake in cold mice. We conclude that CD36 is an important regulator of TEL metabolism and TRL-derived fatty acid uptake into BAT.

Given the high impact of BAT on TRL turnover, we investigated whether BAT activation is also able to lower plasma triglycerides in Apoa5^{-/-} mice. This model of severe hyperlipidemia displays an impaired lipolytic TRL processing^{13,16}. In these mice cold exposure corrected plasma lipids within hours and TRL-triglycerides as well as TRL-cholesterol (FIG. 4a-c) levels declined to values comparable to fasted wild-type mice. Thus, we conclude that modulation of BAT activity can correct hyperlipidemia. To further delineate the biological importance of BAT in a pathophysiological state we analyzed TRL metabolism in a well-established model of diet-induced obesity and insulin resistance (Supplementary table 2)³⁰. Brown adipocytes of obese mice appeared hypertrophic compared to lean controls as determined by environmental scanning electron microscopy (FIG. 4d). Notably, after cold-induced lipolysis, lipid droplets in brown adipocytes from lean and obese mice shrank to a similar extent which was also emphasized by the brownish reappearance of interscapular BAT (FIG. 4e). The expression profile of cold-modulated genes was similar in lean and obese mice (Supplementary FIG. 18) and consequently, there was no significant correlation between body weight and weight loss (Supplementary FIG. 19). Next we investigated whether glucose and TRL metabolism are influenced by BAT in this model of obesity. This is of special Interest, as it was suggested that, in humans, body fat mass inversely correlates with BAT activity as determined by PBT-CT using radioactive glucose tracers^{5,31,32}. However, it so fhr remained unclear whether glucose and/or lipid uptake into BAT is influenced by insulin or insulin resistance^{2,33,34}. As expected, compared to lean controls a combined oral glucose and fat tolerance test displayed an impaired glucose tolerance in control obese mice which was normalized upon cold exposure (Supplementary FIG. 20). Correspondingly, the uptake of ¹⁴C-deoxyglucose (FIG. 4f and Supplementary FIG. 21) and ³H-triolein (FIG. 4g and Supplementary FIG. 22) were significantly increased into BAT of both lean and obese mice. The stimulated glucose uptake might be explained by increased levels of glucose transporters GLUT1 and GLUT4 in BAT and heart (Supplementary FIG. 23). In obese mice glucose uptake into BAT and heart was higher than in lean mice which might be explained by improved local insulin sensitivity in cold mice³³ (Supplementary FIG. 24). Furthermore, TRL clearance was accelerated in obese mice compared to lean controls (FIG. 4h) even when corrected for body weight (Supplementary FIG. 25). Accordingly, we observed a similar uptake of TRL into BAT before and after activation in lean and obese mice when corrected for weights of dissected organs (FIG. 4i and Supplementary FIG. 25) confirming that uptake of TRL into BAT is independent of insulin levels and insulin resistance. Differences in heart uptake of TRL appear to be mouse strain-specific between C57BL/6 and FVB (compare FIG. 2c).

In summary, we show that after short-term cold exposure, BAT is quantitatively important for lipoprotein metabolism. Fatty acids are efficiently channeled into BAT due to a metabolic program that boosts TRL uptake into BAT. This process is associated with increased endothelial permeability for lipoproteins and is crucially dependent on LPL and CD36. BAT activation is able to correct hyperlipidemia and improves deleterious effects of obesity despite insulin resistance. Moreover, we provide a non-invasive method to measure BAT activity using nanocrystals embedded into the lipoprotein core (nanosomes) via MRI. Given the low toxicity of iron-based nanocrystals, this technology can be used in a clinical setting and provides a key tool to assess, e.g., activity of human brown adipose tissue, the future target for therapeutic intervention of obesity and elevated blood lipids.

### Example 2

A method to sense LPL activity by non-invasive magnetic resonance imaging under physiological and pathophysiological conditions in a very high resolution using SPIO-nanosomes

### METHODS

Animals and diets. All experimental procedures were performed with approval from the animal care committees responsible for the University Medical Center Hamburg-Eppendorf. Animals were housed at 22° C. with ad libitum access to standard laboratory chow diet. We used male and female age-matched (16-22 weeks) FVB wildtype mice which were fasted 24 h or 4 h prior to the experiment. Fasting (22° C.) and cold exposure (4° C.) was performed in single cages for 24 h unless indicated otherwise.

For manipulation of LPL function, THL (Roche, 12.5 mg ml⁻¹ DMSO) was diluted to 1.25 mg ml⁻¹ in 10% DMSO in PBS. Mice received 200 μL of either 0.25 mg THL, 50 U heparin (ratiopharm) or PBS (mock). After 1 min, SPIO-nanosomes were injected, and plasma clearance and organ uptake were determined by dynamic MRI.

In vivo imaging studies. MRI was performed as described before¹⁴. Briefly, all static and dynamic MRI measurements were performed with a clinical 3 Tesla MR scanner (Philips Medical Systems, Netherlands) equipped with a custom-made small animal solenoid coil. The dynamic measurements were based on a gradient-echo sequence (Supplementary table 3). The applied sequence is highly sensitive to susceptibility effects caused by local magnetic field inhomogeneities caused by SPIO-TRL. DICOM data were processed with ImageJ (http://rsbweb.nih.gov/ij/). SPIO-nanosomes were injected via a tail vein catheter.

Preparation of TRL comprising SPIO was performed essentially as disclosed in Bruns et al. Nature Nanotechnology 4, 2008:193-201 and the supplement to said publicationn, with the modification that nanoparticles with a size of about 10 nm were used.

### RESULTS

To investigate the LPL as well as lipoprotein clearance and kinetics in more detail, hydrophobic superparamagnetic iron oxide (SPIO) 10 nm sized nanocrystals were embedded into the core of TEL particles. Therefore, 0.1 mg iron in the form of 10 nm SPIO and 5 mg lipids extracted from human TRL lipoproteins were mixed in chloroform. The chloroform was evaporated and 1 ml PBS was added. This mixture was, as described in Bruns et al. Nature Nanotechnology 4, 2008:193201, sonicated for 10 minutes and filtered through a syringe filter. 300 μl of these nanosomes were injected to follow lipoprotein uptake by dynamic magnetic resonance imaging (MRI)¹⁴. Irrespectively of BAT or heart activity, we observed uptake into the liver of control, 24 h fasted and cold-exposed mice. However, 24 h fasting markedly increased the negative contrast of the myocardium and cold exposure markedly increased the negative contrast of several BAT depots, indicative for increased LPL activity.

To assess whether the canonical LPL pathway is involved in uptake of SPIO-nanosomes into BAT, we inhibited LPL activity by injecting tetrahydrolipstatin (THL), a specific inhibitor. Local LPL activity in BAT and heart is required for the uptake of SPIO-nanosomes, as THL pre-treatment abolished uptake of SPIO into the heart of fasted mice and BAT of cold mice (FIGS. 5 and 6).

In addition, the inventors showed that release of LPL from the endothelium by heparin pre-treatment also blocked uptake of SPIO into BAT. It is noteworthy that heparin leads to transient maximized LPL activity in the blood stream. These results indicate that local LPL activity in BAT or the heart drives lipolysis and is required for SPIO-nanosomes uptake into BAT and the heart (FIGS. 5, 6 and 7). The uptake of SPIO-nanosomes into BAT can be blocked by the injection of native chylomicrons. This indicates that the nanosomes used in the method of the invention are recognized by the same machinery (e.g. LPL) as TRL.

Taken together, the experiment shows that uptake of the nanosomes of the invention into BAT and the heart comprises heparin-sensible initial binding to the vessel wall and subsequent internalization of particles in a LPL-dependent manner.

In summary, we demonstrate that it is possible to measure LPL-activity by non-invasive MRI in a very high temporal and spatial resolution using SPIO-nanosomes.

### Example 3

Preparation of Nanosomes comprising SPIO

3A: Extraction of human/patient specific lipid mixtures from TRL

SPIO nanosomes suitable for use in the invention have been prepared by addition of 1 mg dry weight of 6nm or 10 nm SPIO nanocrystals (comprising 0.33 mg iron) to 20 mg human lipid extracted according to methods known in the state of the art, e.g., from the patient.

3B Assembly of nanosomes

40 mg lipids consisting of 78.4% 1,2,3tri-(cis,cis-9,12-octadecadienoyl)glycerol, 19.6% 1,2-diacyl-snglycero-3-phosphocholine, 2% 1-acyl-sn-glycero-3-phosphocholine were mixed and 2 mg dry weight of 6 nm MnFe₂O₄SPIO nanocrystals (0.22 mg iron) added. Micelles were formed according to methods known in the art.

3C Use of Intralipid® for preparation of nanosomes, in particular, SPIO nanosomes

The invention further provides a method for preparing nanosomes, in particular SPIO nanosomes and the nanosomes prepared with this method based on an Intravenous lipid supplement accepted for use in humans, e.g., Intralipid®.

Intralipid®, e.g., Intralipis® 20%, is a 20% intravenous fat emulsion. It is a sterile, non-pyrogenic fat emulsion prepared for intravenous administration as a source of calories and essential fatty acids. It comprises about 20% soybean oil, 1.2% egg yolk phospholipids, 2.25% glycerine and water for injection. Sodium hydroxide has been added to adjust the pH to 6 to 8,9, in particular, 8. The soybean oil may be a refined natural product consisting of a mixture of neutral triglycerides of predominantly unsaturated fatty acids. The major component fatty acids may be linoleic (44-62%), oleic (19-30%), palmitic (7-14%), linolenic (4-11%) and stearic (1.4-5.5%). Methods for lipid extraction and micelle formation are known in the state of the art.

Nanosomes can be prepared from the lipid extracted from this or a similar intravenous lipid supplement accepted for use in humans by addition of 0.25 mg-10 mg (preferably, 0.5 mg-5 mg or 1 mg-3 mg) dry weight of SPIO having a size of 2-30 nm (preferably, 4 nm-16 nm; more preferably 6-10 nm) SPIO nanocrystals to 20 mg human lipid. The nanocrystals preferably comprise about 0.33 mg iron.

This has the advantage that the nanosomes are easily available without using human material, which avoids questions of infection risk and lowers costs.

References

All references cited herein are frilly incorporated by reference.

1. Cannon, B. & Nedergaard, J. Brown adipose tissue: function and physiological significance. Physiol Rev. 84, 277-359 (2004).

2. Enerback, S. Human brown adipose tissue. Cell Metab 11, 248-252 (2010).

3. Cypess, A. M. et al. Identification and importance of brown adipose tissue in adult humans. N. Engl. J. Med 360, 1509-1517 (2009).

4. Virtanen, K. A. et al. Functional brown adipose tissue in healthy adults. N. Engl. J. Med. 360, 1518-1525 (2009).

5. van Marken Lichtenbelt, W. D. et al. Cold-activated brown adipose tissue in healthy men. N. Engl. J. Med. 360, 1500-1508 (2009).

6. Williams, K. J. Molecular processes that handle—and mishandle—dietary lipids. J. Clin. Invest 118, 3247-3259 (2008).

7. Hokanson, J. E. & Austin, M. A. Plasma triglyceride level is a risk factor for cardiovascular disease independent of high-density lipoprotein cholesterol level: a meta-analysis of population-based prospective studies, J. Cardiovasc. Risk 3, 213-219 (1996).

8. Austin, M. A. et al. Cardiovascular disease mortality in familial forms of hypertriglyceridemia: A 20-year prospective study. Circulation 101, 2777-2782 (2000).

9. Cullen, P, Evidence that triglycerides are an independent coronary heart disease risk factor. Am. J. Cardiol. 86, 943-949 (2000).

10. Mooradian, A. D. Dyslipidemia in type 2 diabetes mellitus. Nat. Clin. Pract. Endocrinol. Metab 5, 150-159 (2009).

11. Ginsberg, H. N. Insulin resistance and cardiovascular disease. J. Clin. Invest 106, 453-458 (2000).

12. von, E. A., Hersberger, M. & Rohrer, L. Current understanding of the metabolism and biological actions of HDL. Curr. Opin. Clin. Nutr. Metab Care 8, 147-152 (2005).

13. Merkel, M. et al. Apolipoprotein AV accelerates plasma hydrolysis of triglyceride-rich lipoproteins by interaction with proteoglycan-bound lipoprotein lipase. J. Biol. Chem. 280, 21553-21560 (2005).

14. Bruns, O. T. et al. Real-time magnetic resonance imaging and quantification of lipoprotein metabolism in vivo using nanocrystals. Nat. Nanotechnol. 4, 193-201 (2009).

15. Eiselein, L., Wilson, D. W., Lame, M. W. & Rutledge, J. C. Lipolysis products from triglyceride-rich lipoproteins increase endothelial permeability, perturb zonula occludens-1 and F-actin, and induce apoptosis. Am. J. Physiol Heart Circ. Physiol 292, H2745-H2753 (2007).

16. Pennacchio, L. A. et at. An apolipoprotein influencing triglycerides in humans and mice revealed by comparative sequencing. Science 294, 169-173 (2001).

17. Zhang, S. H., Reddick, R. L., Piedrahita, J. A. & Maeda, N. Spontaneous hypercholesterolemia and arterial lesions in mice lacking apolipoprotein E. Science 258, 468-471 (1992).

18. Brown, M. S. & Goldstein, J. L. A receptor-mediated pathway for cholesterol homeostasis. Science 232, 34-47 (1986).

19. Rohlmann, A., Gotthardt, M., Hammer, R. E. & Herz, J. Inducible Inactivation of hepatic LRP gene by cre-mediated recombination confirms role of LRP in clearance of chylomicron remnants. J. Clin. Invest 101, 689-695 (1998).

20. Beisiegel, U., Weber, W., Ihrke, G., Herz, J. & Stanley, K. K. The LDL-receptor-related protein, LRP, is an apolipoprotein E-binding protein. Nature 341, 162-164 (1989).

21. Gordts, P. L. et al. Inactivation of the LRP1 intracellular NPxYxxL motif in LDLR-deficient mice enhances postprandial dyslipidemla and atherosclerosis. Arterioscler. Thromb. Vasa Biol. 29, 1258-1264 (2009).

22. Augustus, A. S., Kako, Y., Yagyu, H. & Goldberg, I. J. Routes of FA delivery to cardiac muscle: modulation of lipoprotein lipolysis alters uptake of TG-derived FA. Am. J. Physiol Endocrinol. Metab 284, B331-E339 (2003).

23. Neuger, L. et al. Effects of heparin on the uptake of lipoprotein lipase in rat liver. BMC Physiol 4, 13 (2004).

24. Sukonina, V., Lookene, A., Olivecrona, T. & Olivecrona, O. Angiopoietin-like protein 4 converts lipoprotein lipase to inactive monomers and modulates lipase activity in adipose tissue. Proc. Natl. Acad. Sci. U.S.A 103, 17450-17455 (2006).

25. Moore, K. J. et al. Loss of receptor-mediated lipid uptake via scavenger receptor A or CD36 pathways does not ameliorate atherosclerosis in hyperlipidemic mice. J. Clin. Invest 115, 2192-2201 (2005).

26. Goudriaan, J. R. et al CD36 deficiency in mice impairs lipoprotein lipase-mediated triglyceride clearance. J. Lipid Res. 46, 2175-2181 (2005).

27. Carneheim, C., Nedergaard, J. & Cannon, B. Beta-adrenergic stimulation of lipoprotein lipase in rat brown adipose tissue during acclimation to cold. Am. J. Physiol 246, E327-13333 (1984).

28. Hagberg, C. E. et al. Vascular endothelial growth factor B controls endothelial fatty acid uptake. Nature 464, 917-921 (2010).

29. Febbraio, M. et al. A null mutation in murine CD36 reveals an important role in fatty acid and lipoprotein metabolism. J. Biol. Chem. 274, 19055-19062 (1999).

30. Surwit, R. S. et al. Differential effects of fat and sucrose on the development of obesity and diabetes in C57BL/61 and A/J mice. Metabolism 44, 645-651(1995).

31. Saito, M. at al. High incidence of metabolically active brown adipose tissue in healthy adult humans: effects of cold exposure and adiposity. Diabetes 58, 1526-1531 (2009).

32. Zingaretti, M. C. et al. The presence of UCP1 demonstrates that metabolically active adipose tissue in the neck of adult humans truly represents brown adipose tissue. FASEB J. 23, 3113-3120 (2009).

33. Vallerand, A. L., Perusse, F. & Bukowiecki, L. J. Cold exposure potentiates the effect of insulin on in vivo glucose uptake. Am. J. Physiol 253, E179-E186 (1987).

34. Skarulis, M. C. et al. Thyroid hormone induced brown adipose tissue and amelioration of diabetes in a patient with extreme insulin resistance. J. Clin. Endocrinol. Metab 95, 256-262 (2010).

35. Dole, V. P. A relation between non-esterified fatty acids in plasma and the metabolism of glucose. J. Clin. Invest 35, 150-154 (1956).

36. Hohenberg, H., Tobler, M. & Muller, M. High-pressure freezing of tissue obtained by fine-needle biopsy. J. Microsc. 183, 133-139 (1996).

37. Alivisatos P. The use of nanocrystals in biological detection. Nat Biotechnol. 2004 January;22(1):47-52.

38. Augustus A S, Buchanan J, Park T S, Hirata K, Nob H L, Sun J, Homma S, D'armiento J, Abel E D, Goldberg U. Loss of lipoprotein lipase-derived fatty acids leads to increased cardiac glucose metabolism and heart dysfunction. J Biol Chem. 2006 Mar 31;281(13):8716-23.

39. Bruns O T, Ittrich H, Peldschus K, Kaul M G, Tromsdorf U I, Lauterwasser J, Nikolic M S, Mollwitz B, Merkel M, Bigall N C, Sapra S, Reimer R, Hohenberg Weller H, Eychmüller A, Adam O, Beisiegel U, Heeren J. Real-time magnetic resonance imaging and quantification of lipoprotein metabolism in vivo using nanocrystals. Nat Nanotechnol. 2009 March;4(3):193-201.

40. Heintel D, Kienle D, Shehata M, Krbber A, Kroemer E, Schwarzinger I, Mitteregger D, Le T, Gleiss A, Mannhalter C, Chott A, Schwarzmeier J, Fonatsch C, Gaiger A, Döhner H, Stilgenbauer S, Jäger U; CLL Study Group. High expression of lipoprotein lipase in poor risk B-cell chronic lymphocytic leukemia. Leukemia. 2005 July;19(7):1216-23.

41. Hong R, Cima M J, Weissleder R, Josephson L. Magnetic microparticle aggregation for viscosity determination by MR. Magn Reson Med. 2008 March;59(3):515-20.

42. Koh I, Hong R, Weissleder R, Josephson L. Sensitive NMR sensors detect antibodies to influenza. Angew Chem Int Ed Engl, 2008;47(22):4119-21.

43. Lee H, Sun E, Ham D, Weissleder R. Chip-NMR biosensor for detection and molecular analysis of cells. Nat Med. 2008 August;14(8):869-74.

44. MoLaurin E J, Greytak A B, Bawendi M G, Nocera D O. Two-photon absorbing nanocrystal sensors for ratiometric detection of oxygen. J Am Chem Soc. 2009 Sep. 16;131(36):12994-3001.

45. Merkel M, Eckel R H, Goldberg I J. Lipoprotein lipase: genetics, lipid uptake, and regulation. J Lipid Res. 2002 December;43(12):1997-2006/

46. Medintz I L, Uyeda H T, Goldman E R, Mattoussi H. Quantum dot bioconjugates for imaging, labelling and sensing. Nat Mater. 2005 June;4(6):435-46.

47. Michalet X, Pinaud F F, Bentolila L A, Tsay J M, Doose S, Li JJ, Sundaresan G, Wu A M, Gambhir S S, Weiss S. Quantum dots for live cells, in vivo imaging, and diagnostics. Science. 2005 Jan. 28;307(5709):538-44.

48. Murray C B, Norris D J, Bawendi M G. Synthesis and characterization of nearly monodisperse CdE (E=sulfur, selenium, tellurium) semiconductor nanocrystallites J. Am. Chem. Soc., 1993, 115 (19), pp 8706-8715

49. Olivecrona O., Olivecrona T. Triglyceride lipases and atherosclerosis. Curr Opin Lipidol. 2010 Jul. 30. [Epub ahead of print]

50. Oppezzo P, Vasconcelos Y, Settegrana C, Jeannel D, Vuillier F, Legarff-Tavernier M, Kimura EY, Bechet S, Dumas O, Brlssard M, Merle-Béral H, Yamamoto M, Dighiero G, Davi F; French Cooperative Group on CLL. The LPL/ADAM29 expression ratio is a novel prognosis indicator in chronic lymphocytic leukemia. Blood. 2005 Jul. 15;106(2):650-7.

51. Park T S, Yamashita H, Blaner W S, Goldberg I J. Lipids in the heart: a source of fret and a source of toxins. Curr Opin Lipidol. 2007 June;18(3):277-82.

52. Perez J M, Josephson L, O'Loughlin T, Högemann D, Weissleder R. Magnetic relaxation switches capable of sensing molecular interactions. Nat Biotechnol. 2002 August;20(8):816-20.

53. Snee P T, Somers R C, Nair G, Zimmer J P, Bawendi M G, Nocera D G. A ratiometric CdSe/ZnS nanocrystal pH sensor. J Am Chem Soc. 2006 Oct. 18;128(41):13320-1.

54. Trost Z, Sok M, Marc J, Cerne D. Increased lipoprotein lipase activity in non-small cell lung cancer tissue predicts shorter patient survival. Arch Med Res. 2009 July;40(5):364-8.

55. Williams K J. Molecular processes that handle—and mishandle—dietary lipids. J Clin Invest. 2008 October;118(10):3247-59.

56, Yamashita H, Bharadwaj K G, Ikeda S, Park T S, Goldberg I J. Cardiac metabolic compensation to hypertension requires lipoprotein lipase. Am J Physiol Endocrinol Metab. 2008 September;295(3):E705-13.

57. Frias, J. C., Williams, K. J., Fisher, E. A. & Fayad, Z. A. Recombinant HDL-like nanoparticles: a specific contrast agent for MRI of atherosclerotic plaques. 3. Am. Chem. Soc. 126, 16316-16317 (2004).

58. Corbin, I. R. et al. Low-density lipoprotein nanoparticles as magnetic resonance imaging contrast agents. Neoplasia 8, 488-498 (2006).

59. Glickson, J. D. et al. Lipoprotein nanoplatform for targeted delivery of diagnostic and therapeutic agents. Mol. Imaging 7, 101-110 (2008).

60. Tromsdorf et al., 2007, Nano Letters 7, 2422-2427.

1. A method of in vivo determining and/or visualizing lipoprotein lipase (LPL) activity, comprising
a) administering a composition comprising superparamagnetic iron oxide nanocrystals (SPIO) incorporated in the core of nanosomes such as lipoproteins to a subject, and
b) determining presence of SPIO in a compartment of the subject, wherein the presence of SPIO indicates lipoprotein lipase (LPL) activity in the compartment.

2. The method of claim 1, wherein the LPL activity is presented as an image.

3. The method of claim 1, wherein the compartment is an organ, a tissue or a cell or an area thereof.

4. The method of claim 3, wherein the organ is selected from the group consisting of heart, skeletal muscle and brain.

5. The method of claim 1, wherein the visualization is visualization of an organ, a tissue or a cell.

6. The method of claim 1, wherein the administration is by oral or intravenous administration.

7. The method of claim 1, wherein the determination is performed by MRI or by Magnetic Particle Imaging (MPI).

8. The method of claim 1, wherein the SPIO comprise nanocrystals having a size of about 2 to about 20 nm.

9. The method of claim 1, wherein the method is non-invasive and/or does not require administration of radioactive compounds to the subject.

10. The method of claim 3, wherein the tissue is tumor tissue and the LPL activity is predictive of progression of the tumor, in particular, wherein a high LPL activity is predictive of fast progression of the tumor.

11. The method of claim 3, wherein the organ is heart and wherein scarring due to infarction is detected.

12. The method of claim 3, wherein the organ is heart and wherein disorders of the lipid metabolism are detected.

13. A method of predicting clinical outcome for a tumor, comprising performing the method of claim 1, wherein a high lipase activity correlates with fast progression of the tumor and a low lipase activity correlates with slow progression or regression of the tumor.

14. A method of diagnosing a disorder of the lipid metabolism, comprising performing the method of claim 1.

15. A method of diagnosing a cardiac disorder, comprising performing the method of any of claim 1.

16. The method of claim 13, comprising comparing the results with results from a healthy subject and/or a from a group of healthy subjects and/or a subject or subjects previously diagnosed with said disorder or tumor.

17. (canceled)

18. (canceled)

19. A method for preparing nanosomes, comprising extracting lipids from an intravenous lipid supplement accepted for use in humans, and mixing them with SPIO nanocrystals.

20. Nanosomes prepared according to the method of claim 19.

21. The method of claim 3, wherein the tissue is selected from the group consisting of tumor, atherosclerotic plaque and adipose tissue.

22. The method of claim 21, wherein the adipose tissue is white adipose tissue or brown adipose tissue.

23. The method of claim 6, wherein the administration is by intravenous administration.

24. The method of claim 7, wherein the determination is performed by dynamic MRI.

25. The method of claim 8, wherein the SPIO comprise nanocrystals having a size of about 6 to about 10 nm.

26. The method of claim 1, wherein SPIO comprise Fe₃O₄ nanocrystals.

27. The method of claim 10, wherein a high LPL activity is predictive of fast progression of the tumor.

28. The method of claim 14, comprising comparing the results with results from a healthy subject and/or a from a group of healthy subjects and/or a subject or subjects previously diagnosed with said disorder or tumor.

29. The method of claim 15, comprising comparing the results with results from a healthy subject and/or a from a group of healthy subjects and/or a subject or subjects previously diagnosed with said disorder or tumor.

30. The method of claim 19, wherein the nanosomes are SPIO nanosomes.
